(19) United States
(12) Patent Application Publication
(10) Pub. No.: US 2026/0193725 A1
(43) Pub. Date: Jul. 9, 2026

(54) Label-Free Detection of Oligonucleotide Hybridization Using Anchored Single-Walled Carbon Nanotube Corona Phase

(71) Applicant: Massachusetts Institute of Technology, Cambridge, MA (US)

(72) Inventors: Michael S Strano, Lexington, MA (US); Jianqiao Cui, Cambridge, MA (US); Xun Gong, Boston, MA (US); Xiaojia Jin, Cambridge, MA (US); Sungyun Yang, Cambridge, MA (US); Sooyeon Cho, Boston, MA (US)

(21) Appl. No.: 19/133,159

(22) PCT Filed: Nov. 30, 2023
(86) PCT No.: PCT/US2023/081973

§ 371 Date: May 27, 2025

Related U.S. Application Data
(60) Provisional application No. 63/385,557, filed on Nov. 30, 2022.

(51) Int. Cl. C12Q 1/70; B82Y 15/00; B82Y 20/00; C09K 11/02; C09K 11/65; C12Q 1/6874

(52) U.S. Cl. CPC C12Q 1/701; C09K 11/025; C09K 11/65; C12Q 1/6874; B82Y 15/00; B82Y 20/00

(57) ABSTRACT

The present invention provides a system and method for detecting an analyte in a sample comprising semiconducting single-walled carbon nanotubes (SWCNTs) and surface-adsorbed nucleic acids, wherein the surface-adsorbed nucleic acids comprise a complementary region and at least one anchor, wherein the complementary region comprises a nucleic acid sequence that is complementary to and/or hybridizes to a target region of the analyte, and the at least one anchor comprises a nucleic acid sequence that is not complementary to the target region of the analyte.

### CROSS REFERENCE TO RELATED APPLICATIONS

This application claims priority to U.S. Provisional Patent Application No. 63/385,557, filed Nov. 30, 2022, the contents of which are hereby incorporated by reference in their entirety.

### STATEMENT REGARDING FEDERALLY SPONSORED RESEARCH OR DEVELOPMENT

This invention was made with government support under 1R42DE030829 awarded by National Institutes of Health. The government has certain rights in this invention.

### REFERENCE TO AN ELECTRONIC SEQUENCE LISTING

The instant application contains a Sequence Listing which has been submitted as an electronic sequence file and is hereby incorporated by reference in its entirety. The name of the file is “23-1481-WO_SequenceListing.xml,” it was created on Nov. 30, 2023, and is 75,800 bytes in size.

### BACKGROUND

Detection of single-stranded oligonucleotides through complementary hybridization plays an essential role in diagnostic pathology,^{1,2} drug discovery and delivery,^{3,4} and molecular biology.^{5,6} In the case of biosensing, materials with unique nanoscale properties were often used as scaffolds for single-stranded DNA (ssDNA) immobilization to construct probe or transducer elements,⁷ with specificity arising from hybridization at the solid-liquid nanomaterial interface. For these constructs, interfacial traits including surface strand density,⁸⁻¹² surface charge,¹³⁻¹⁵ substrate porosity,¹⁶ point mismatch,¹⁷⁻¹⁹ immobilized DNA length,²⁰ brush effect for long targets²¹ and probe attachment chemistry²²⁻²⁴ have been shown to influence hybridization stability and kinetics. While initial studies involved flat microchip arrays, nanomaterials such as gold nanoparticles^{22,25-28} and carbon nanotubes²⁹⁻³² revealed unique interfacial properties potentially useful for sensor design. Such nanomaterial surfaces often entail constrained probe conformations that result in noncanonical hybridization behaviors that are not as well studied.

A large fraction of current DNA-nanomaterial hybrid studies utilize carbon-based substrates.³³⁻³⁷ Of them, semiconducting single-walled carbon nanotubes (SWCNTs) have advantages as sensor components, particularly due to their photophysical properties.^{38,39} Small diameter SWCNTs are favorable for biomedical applications because of their photoluminescence (PL) emissions at near-infrared (nIR) wavelengths,⁴⁰ where there is minimal optical absorption from blood and tissue⁴¹⁻⁴³ as well as cellular autofluorescence.⁴⁴ Additionally, SWCNTs are resistant to photobleaching^{45,46} compared to conventional fluorescent dyes, allowing for long-term temporal monitoring of fluorescence signals.⁴⁶⁻⁴⁷ Furthermore, SWCNTs can be engineered to form specifically tailored and stable corona phases (CPs),⁴⁸ or non-covalent wrappings, in aqueous environments for specific analyte recognition with up to single-molecule sensitivity.⁴⁹⁻⁵⁰ When a target analyte binds to the SWCNT corona, the perturbation introduced to the local environment can be transduced via changes in the SWCNT fluorescence signal in the form of modulations in their emission wavelength⁵¹ and/or intensity.⁵²

The ssDNA SWCNT CPs have been explored recently as hybridization sensors against DNA (ssDNA),⁵³ microRNA,³¹ and viral RNA targets.^{32,54} These sensors report local target analyte concentrations in the form of PL emission wavelength shifts. Some studies have included non-complementary ssDNA regions within the CP to improve hybridization signal transduction, the theory being that having “anchor” regions will allow for increase solution-phase analyte interactions.⁵⁵ Although these anchor regions have shown promise, their design is not well understood. Furthermore, it is well known that ssDNA as part of the SWCNT CP behaves non-canonically, making hybridization efficacy sequence dependent. Nevertheless, complementary sequences have been shown to be correlated with corona phase reorganization as demonstrated by atomic force microscopy of 30-mer oligonucleotides.⁵⁶ However, differences between corona phase sequences prompt a systematic study of the ssDNA-SWCNT hybridization process to enable directed search of sensor candidates.

### SUMMARY OF THE INVENTION

In one aspect, the present disclosure provides a system for detecting an analyte in a sample comprising semiconducting single-walled carbon nanotubes (SWCNTs) and surface-adsorbed nucleic acids, wherein the surface-adsorbed nucleic acids comprise a complementary region and at least one anchor, wherein the complementary region comprises a nucleic acid sequence that is complementary to and hybridizes to a target region of the analyte and wherein the at least one anchor comprises a nucleic acid sequence that is not complementary to the target region of the analyte.

In a second aspect, the present disclosure provides a method of detecting an analyte in a sample, the method comprising providing a system according to the first aspect of the disclosure in a buffer solution, combining the sample with the system, incubating the system and the sample, and measuring the photoluminescence (PL) of the system, wherein a shift in wavelength and/or intensity of the system relative to the system without exposure to analyte indicates the presence of the analyte.

The following Detailed Description references the accompanying drawings which form a part this application, and which show, by way of illustration, specific example implementations. Other implementations may be made without departing from the scope of the disclosure.

### BRIEF DESCRIPTION OF THE DRAWINGS

FIG. 1. Single-walled carbon nanotube (SWCNT) with single-stranded DNA (ssDNA) corona for the detection of viral nucleic acid through hybridization. (FIG. 1A) Schematic of the ssDNA functionalized SWCNT for detecting target nucleic acid (analyte). Upon incubation, the target nucleic acid in solution hybridizes with the adsorbed ssDNA strands on SWCNT surface, resulting in a measurable wavelength modulation of the SWCNT fluorescence signal. (FIG. 1B) An example of observed hypsochromic shifts in the PL spectra of the DNA-SWCNT construct near 990 nm upon incubation with the target complementary analyte. No shift was observed in non-complementary control and PBS buffer. (FIG. 1C) Kinetics of the SWCNT 990 nm peak optical transition energy shift (E_{Analyte}−E₀) upon incubation with cDNA or nDNA at 24° C., 37° C. or 50° C., fitted with the Langmuir adsorption model. E_{Analyte} and E₀ denote the transition energy after and before analyte addition respectively. Data presented as mean±s.d., n=3. (FIG. 1D) Arrhenius plots of the hybridization and denaturation rate constants obtained from the Langmuir adsorption model. The R-squared values for the fittings for k_{f} (dashed) and k_{b} (solid) are 0.979 and 0.976 respectively. (FIG. 1E) Normalized change in DNA surface coverage of SWCNT over time upon incubation with cDNA or nDNA at 37° C. or 50° C. Specific adsorption occurs in the presence of cDNA but not nDNA.

FIG. 2: Influence of anchor in adsorbed strand on SWCNT wavelength modulation and surface coverage. (FIG. 2A) Responses of 11 ssDNA-SWCNT complexes toward 11 unique sequences from SARS-CoV-2 in Table 1. The ssDNA used to functionalize SWCNT contains only the complementary region without an anchor region. Both DNA (top panel) and RNA (bottom panel) complementary and non-complimentary analytes elicited mainly bathochromic shifts. (FIG. 2B) Responses of S1 ssDNA-SWCNT complexes with (AT)₁₅, (CT)₁₅, or (GT)₁₅ anchor and without anchor toward DNA (top panel) and RNA (bottom panel) analytes. The anchor was added to either the 5′ or the 3′ end of the complementary region. SWCNT PL responses toward nDNA and nRNA were significantly suppressed for constructs with an anchor region. (FIG. 2C) Responses of S1 ssDNA-SWCNT complexes with (CT)ₓ (left panel) or (GT)ₓ (right panel) anchor of different lengths (x=10, 15, 20) toward DNA (top panel) and RNA (bottom panel) analytes. All data presented as mean±s.d., n=3. (FIG. 2D) Probability distribution of normalized surface coverage changes of 11 SWCNT constructs without anchors in (a) upon DNA (left panel) and RNA (right panel) analytes introduction. (FIG. 2E) Probability distribution of normalized surface coverage change of S1 SWCNT constructs with anchors in (b) upon DNA (left panel) and RNA (right panel) analytes introduction. (FIG. 2F) Probability distribution of normalized surface coverage change of S1 SWCNT constructs with (CT)ₓ and (GT)ₓ anchors in (c) upon DNA (left panel) and RNA (right panel) analytes introduction.

FIG. 3: Screening results of the ssDNA-SWCNT library targeting 11 sequences. (FIG. 3A) Solvatochromic shifts and (FIG. 3B) normalized change in surface coverage by DNA upon incubation with 500 nM cDNA and cRNA. For each target sequence, 4 anchor regions were used, namely (CT)₁₅ attached at 5′ end, (CT)₁₅ attached at 3′ end, (GT)₁₅ attached at 5′ and (GT)₁₅ attached at 3′ end of the complementary region. Red denotes a bathochromic shift in (a) and increase in surface coverage in (b), while blue denotes a hypsochromic shift in (a) and decrease in surface coverage in (b). The heat maps showing responses toward nDNA and nRNA controls are given in the FIG. S1. All data presented as mean, n=3. (FIG. 3C) Scatter plots of normalized surface coverage change against optical transition energy change for CPs with and without anchors upon addition of DNA (left plot) and RNA analytes (right plot). (FIG. 3D) Correlations between normalized surface coverage change after cDNA addition and z-scored initial surface coverage before cDNA addition. A statistically significant negative correlation with Pearson coefficient of −0.527 (P<0.001) was observed for anchored CP denoted by the blue crosses. No meaningful correlation was observed for unanchored CPs denoted by the green crosses.

FIG. 4: Influence of incubation conditions on sensor solvatochcromic responses and surface coverage change. (a-c) M2-(CT)₁₅-SWCNT solvatochromic responses when bath sonication was carried out (FIG. 4A) after the 1-hour incubation at 37° C., (FIG. 4B) before the 1-hour incubation at 37° C., or (FIG. 4C) before the 1-hour incubation at room temperature. Conditions are listed below (FIG. 4D). (FIG. 4D) Surface coverage of M2-(CT)₁₅-SWCNT in (a-c) normalized to the initial surface coverage without sonication (PBS addition, 0 min sonication). (FIG. 4E) Solvatochromic responses when SDBS was added to the dispersion concurrently with the analyte before incubation. (FIG. 4F) Response when SDS was added concurrently with the analyte before incubation. (FIG. 4G) Response when SDS was added to the SWCNT dispersion 12 hours before the analyte was added. Amphiphilic molecules decreased the response specificity. Gray shaded regions represent surfactant concentration ranges beyond which response specificity started to worsen. Conditions are listed below. All data presented as mean±s.d., n=3.

FIG. 5: Summary of the best constructs for detecting complementary DNA and RNA targets, their detection limits, specificity, and responses in biofluids. (FIG. 5A) Responses of the best 2 DNA and best 2 RNA detecting constructs against 500 nM complementary and non-complementary analytes. (FIG. 5B) Calibration curves showing energy shifts toward 0.1 nM to 1000 nM analytes fitted to Langmuir binding model to quantify the kinetics parameters. Fitting parameters are α=2.692±0.7, 2.261±0.285, 1.92±0.54, and 0.833±0.247 meV, and K_{D}=150.8±148, 181.3±81.2, 41±64.6, and 67.3±97.6 nM for M2-(CT)₁₅, (CT)₁₅-E16, N1-(GT)₁₅ and S3-(GT)₁₅, respectively. (FIG. 5C) Heatmap showing the responses toward one complementary target sequence and ten non-complimentary sequences at 500 nM. Red: bathochromic shift, blue: hypsochromic shift. Responses demonstrate that complementary target-induced hypsochromic shift or increase in energy is specific over non-complimentary sequences. (FIG. 5D) Responses of N1-(GT)₁₅ and S3-(GT)₁₅ toward 500 nM cRNA and nRNA in PBS buffer and 1% saliva. Solvatochromic shifts when the ssDNA-SWCNT construct was diluted in PBS buffer and 1% saliva do not show significant difference using a two-sample t-test analysis (P=0.127 for N1-(GT)₁₅ and P=0.0573 for S3-(GT)₁₅). NS: not significant. All data presented as mean±s.d., n=3.

FIG. 6: (FIG. 6A) Solvatochromic shifts and (FIG. 6B) normalized surface coverage change of the ssDNA-SWCNT library against 500 nM nDNA and nRNA. Red denotes a bathochromic shift in (FIG. 6A) and increase in surface coverage in (FIG. 6B), while blue denotes a hypsochromic shift in (a) and decrease in surface coverage in (b). All data presented as mean, n=3.

FIG. 7: Correlations between normalized surface coverage change after nDNA addition and initial surface coverage before nDNA addition. No statistically significant correlation was observed for anchored CP and unanchored CPs.

FIG. 8: Solvatochromic responses of the best 2 DNA sensors toward ssDNA and dsDNA analytes. Sensor responses toward ds cDNA and ss nDNA are comparable, indicating no hybridization with the dsDNA targets. All data presented as mean±s.d., n=3.

FIG. 9: Sensor solvatochromic shift response toward cDNA (FIGS. 9A-9E) and cRNA (FIGS. 9F-9J) against the maximum (most negative) free energy of hairpin formation, ΔG_{Hp}, among all potential hairpins of the target sequences. Responses of 5′ (CT)₁₅ anchored CP (FIG. 9A, FIG. 9F), 5′ (GT)₁₅ anchored CP (FIG. 9B, FIG. 9G), 3′ (CT)₁₅ anchored CP (FIG. 9C, FIG. 9H), 3′ (GT)₁₅ anchored CP (FIG. 9D, FIG. 9I) and averaged responses from 4 types of CPs (FIG. 9E, FIG. 9J). Pearson correlation coefficients and p-values are indicated.

FIG. 10: Normalized nanotube surf ace coverage toward cDNA (FIGS. 10A-10E) and cRNA (FIGS. 10f-10j) against the maximum (most negative) free energy of hairpin formation, ΔG_{Hp}, among all potential hairpins of the target sequences. Responses of 5′ (CT)₁₅ anchored CP (FIG. 10A, FIG. 10F), 5′ (GT)₁₅ anchored CP (FIG. 10B, FIG. 10G), 3′ (CT)₁₅ anchored CP (FIG. 10C, FIG. 10H), 3′ (GT)₁₅ anchored CP (FIG. 10D, FIG. 10I) and averaged responses from 4 types of CPs (FIG. 10E, FIG. 10J). Pearson correlation coefficients and p-values are indicated.

FIG. 11: Sensor solvatochromic shift response toward cDNA (FIGS. 11A-11E) and cRNA (FIGS. 11F-11J) against the percent length of the shortest single-stranded section among all hairpins, L_{Hp}, of the target sequences. Responses of 5′ (CT)₁₅ anchored CP (FIG. 11A, FIG. 11F), 5′ (GT)₁₅ anchored CP (FIG. 11B, FIG. 11G), 3′ (CT)₁₅ anchored CP (FIG. 11C, FIG. 11H), 3′ (GT)₁₅ anchored CP (FIG. 11D, FIG. 11I) and averaged responses from 4 types of CPs (FIG. 11E, FIG. 11J). Pearson correlation coefficients and p-values are indicated.

FIG. 12: Normalized nanotube surface coverage change toward cDNA (FIGS. 12A-12E) and cRNA (FIGS. 12-12J) against the percent length of the shortest single-stranded section among all hairpins, L_{Hp}, of the target sequences. Responses of 5′ (CT)₁₅ anchored CP (FIG. 12A, FIG. 12F), 5′ (GT)₁₅ anchored CP (FIG. 12B, FIG. 12G), 3′ (CT)₁₅ anchored CP (FIG. 12C, FIG. 12H), 3′ (GT)₁₅ anchored CP (FIG. 12D, FIG. 12I) and averaged responses from 4 types of CPs (FIG. 12E, FIG. 12J). Pearson correlation coefficients and p-values are indicated.

FIG. 13: Sensor solvatochromic shift response toward cDNA (FIGS. 13A-13E) and cRNA (FIGS. 13F-13J) against the free energy of hybridization, ΔG_{Hyb}, of the target sequences. Responses of 5′ (CT)₁₅ anchored CP (FIG. 13A, FIG. 13F), 5′ (GT)₁₅ anchored CP (FIG. 13B, FIG. 13G), 3′ (CT)₁₅ anchored CP (FIG. 13C, FIG. 13H), 3′ (GT)₁₅ anchored CP (FIG. 13D, FIG. 13I) and averaged responses from 4 types of CPs (FIG. 13E, FIG. 13J). Pearson correlation coefficients and p-values are indicated.

FIG. 14: Normalized nanotube surface coverage change toward cDNA (FIGS. 14A-14E) and cRNA (FIGS. 14F-14J) against the free energy of hybridization, ΔG_{Hyb}, of the target sequences. Responses of 5′ (CT)₁₅ anchored CP (FIG. 14A, FIG. 14F), 5′ (GT)₁₅ anchored CP (FIG. 14B, FIG. 14G), 3′ (CT)₁₅ anchored CP (FIG. 14C, FIG. 14H), 3′ (GT)₁₅ anchored CP (FIG. 14D, FIG. 14I) and averaged responses from 4 types of CPs (FIG. 14E, FIG. 14J). Pearson correlation coefficients and p-values are indicated.

FIG. 15: Sensor solvatochromic shift response toward cDNA (FIGS. 15A-15E) and cRNA (FIGS. 15F-15J) against the maximum (most negative) free energy of self-dimerization, ΔG_{Dm}, among all potential self-dimers of the target sequences. Responses of 5′ (CT)₁₅ anchored CP (FIG. 15A, FIG. 15F), 5′ (GT)₁₅ anchored CP (FIG. 15B, FIG. 15G), 3′ (CT)₁₅ anchored CP (FIG. 15C, FIG. 15H), 3′ (GT)₁₅ anchored CP (FIG. 15D, FIG. 15I) and averaged responses from 4 types of CPs (FIG. 15E, FIG. 15J). Pearson correlation coefficients and p-values are indicated.

FIG. 16: Normalized nanotube surface coverage change toward cDNA (FIGS. 16A-16E) and cRNA (FIGS. 16F-16J) against the maximum (most negative) free energy of self-dimerization, ΔG_{Dm}, among all potential self-dimers of the target sequences. Responses of 5′ (CT)₁₅ anchored CP (FIG. 16A, FIG. 16F), 5′ (GT)₁₅ anchored CP (FIG. 16B, FIG. 16G), 3′ (CT)₁₅ anchored CP (FIG. 16C, FIG. 16H), 3′ (GT)₁₅ anchored CP (FIG. 16D, FIG. 16I) and averaged responses from 4 types of CPs (FIG. 16E, FIG. 16J). Pearson correlation coefficients and p-values are indicated.

FIG. 17: Sensor solvatochromic shift response toward cDNA (FIGS. 17A-17E) and cRNA (FIGS. 17F-17J) against the percent length of the single-stranded section of the self-dimer with the maximum ΔG_{Dm}, L_{Dm}, of the target sequences. Responses of 5′ (CT)₁₅ anchored CP (FIG. 17A, FIG. 17F), 5′ (GT)₁₅ anchored CP (FIG. 17B, FIG. 17G), 3′ (CT)₁₅ anchored CP (FIG. 17C, FIG. 17H), 3′ (GT)₁₅ anchored CP (FIG. 17D, FIG. 17I) and averaged responses from 4 types of CPs (FIG. 17E, FIG. 17J). Pearson correlation coefficients and p-values are indicated.

FIG. 18: Normalized nanotube surface coverage change toward cDNA (FIGS. 18A-18E) and cRNA (FIGS. 18F-18J) against the percent length of the single-stranded section of the self-dimer with the maximum ΔG_{Dm}, L_{Dm}, of the target sequences. Responses of 5′ (CT)₁₅ anchored CP (FIG. 18A, FIG. 18F), 5′ (GT)₁₅ anchored CP (FIG. 18B, FIG. 18G), 3′ (CT)₁₅ anchored CP (FIG. 18C, FIG. 18H), 3′ (GT)₁₅ anchored CP (FIG. 18D, FIG. 18I) and averaged responses from 4 types of CPs (FIG. 18E, FIG. 18J). Pearson correlation coefficients and p-values are indicated.

FIG. 19: Sensor solvatochromic shift response toward cDNA (FIGS. 19A-19E) and cRNA (FIGS. 19F-19J) against the minimum percent length of the single-stranded section of all self-dimers and hairpins, L_{Min} of the target sequences. Responses of 5′ (CT)₁₅ anchored CP (FIG. 19A, FIG. 19F), 5′ (GT)₁₅ anchored CP (FIG. 19B, FIG. 19G), 3′ (CT)₁₅ anchored CP (FIG. 19C, FIG. 19H), 3′ (GT)₁₅ anchored CP (FIG. 19D, FIG. 19I) and averaged responses from 4 types of CPs (FIG. 19E, FIG. 19J). Pearson correlation coefficients and p-values are indicated.

FIG. 20: Normalized nanotube surface coverage change toward cDNA (FIGS. 20A-20E) and cRNA (FIGS. 20F-20J) against the minimum percent length of the single-stranded section of all self-dimers and hairpins, L_{Min} of the target sequences. Responses of 5′ (CT)₁₅ anchored CP (FIG. 20A, FIG. 20F), 5′ (GT)₁₅ anchored CP (FIG. 20G, FIG. 20G), 3′ (CT)₁₅ anchored CP (FIG. 20C, FIG. 20H), 3′ (GT)₁₅ anchored CP (FIG. 20D, FIG. 20I) and averaged responses from 4 types of CPs (FIG. 20E, FIG. 20J). Pearson correlation coefficients and p-values are indicated.

FIG. 21: Sensor solvatochromic shift response toward cDNA (FIGS. 21A-21E) and cRNA (FIGS. 21F-21J) against the number self-dimers formed by the target sequences, N_{Dm}. Responses of 5′ (CT)₁₅ anchored CP (FIG. 21A, FIG. 21F), 5′ (GT)₁₅ anchored CP (FIG. 21B, FIG. 21G), 3′ (CT)₁₅ anchored CP (FIG. 21D, FIG. 21H), 3′ (GT)₁₅ anchored CP (FIG. 21D, FIG. 21I) and averaged responses from 4 types of CPs (FIG. 21E, FIG. 21J). Pearson correlation coefficients and p-values are indicated.

FIG. 22: Normalized nanotube surface coverage change toward cDNA (FIGS. 22A-22E) and cRNA (FIGS. 22F-22J) against the number self-dimers formed by the target sequences, N_{Dm}. Responses of 5′ (CT)₁₅ anchored CP (FIG. 221A, FIG. 22F), 5′ (GT)₁₅ anchored CP (FIG. 221B, FIG. 22G), 3′ (CT)₁₅ anchored CP (FIG. 221C, FIG. 22H), 3′ (GT)₁₅ anchored CP (FIG. 221D, FIG. 22I) and averaged responses from 4 types of CPs (FIG. 22E, FIG. 22J). Pearson correlation coefficients and p-values are indicated.

FIG. 23: Sensor solvatochromic shift response toward cDNA (FIGS. 23A-23E) and cRNA (FIGS. 23F-23J) against adenine content of the target sequences. Responses of 5′ (CT)₁₅ anchored CP (FIG. 23A, FIG. 23F), 5′ (GT)₁₅ anchored CP (FIG. 23B, FIG. 23G), 3′ (CT)₁₅ anchored CP (FIG. 23C, FIG. 23H), 3′ (GT)₁₅ anchored CP (FIG. 23D, FIG. 23I) and averaged responses from 4 types of CPs (FIG. 23E, FIG. 23J). Pearson correlation coefficients and p-values are indicated.

FIG. 24: Normalized nanotube surface coverage change toward cDNA (FIGS. 24A-24E) and cRNA (FIGS. 24F-24J) against adenine content of the target sequences. Responses of 5′ (CT)₁₅ anchored CP (FIG. 24A, FIG. 24F), 5′ (GT)₁₅ anchored CP (FIG. 24B, FIG. 24G), 3′ (CT)₁₅ anchored CP (FIG. 24C, FIG. 24H), 3′ (GT)₁₅ anchored CP (FIG. 24D, FIG. 24I) and averaged responses from 4 types of CPs (FIG. 24E, FIG. 24J). Pearson correlation coefficients and p-values are indicated.

FIG. 25: Sensor solvatochromic shift response toward cDNA (FIGS. 25A-25E) and cRNA (FIGS. 25F-25J) against thymine or uracil content of the target sequences. Responses of 5′ (CT)₁₅ anchored CP (FIG. 25A, FIG. 25F), 5′ (GT)₁₅ anchored CP (FIG. 25B, FIG. 25G), 3′ (CT)₁₅ anchored CP (FIG. 25C, FIG. 25H), 3′ (GT)₁₅ anchored CP (FIG. 25D, FIG. 25I) and averaged responses from 4 types of CPs (FIG. 25E, FIG. 25J). Pearson correlation coefficients and p-values are indicated.

FIG. 26: Normalized nanotube surface coverage change toward cDNA (FIGS. 26A-26E) and cRNA (FIGS. 26F-26J) against thymine or uracil content of the target sequences. Responses of 5′ (CT)₁₅ anchored CP (FIG. 26A, FIG. 26F), 5′ (GT)₁₅ anchored CP (FIG. 26B, FIG. 26G), 3′ (CT)₁₅ anchored CP (FIG. 26C, FIG. 26H), 3′ (GT)₁₅ anchored CP (FIG. 26D, FIG. 26I) and averaged responses from 4 types of CPs (FIG. 26E, FIG. 26J). Pearson correlation coefficients and p-values are indicated.

FIG. 27: Sensor solvatochromic shift response toward cDNA (FIGS. 27A-27E) and cRNA (FIGS. 27F-27J) against cytosine content of the target sequences. Responses of 5′ (CT)₁₅ anchored CP (FIG. 27A, FIG. 27F), 5′ (GT)₁₅ anchored CP (FIG. 27B, FIG. 27G), 3′ (CT)₁₅ anchored CP (FIG. 27C, FIG. 27H), 3′ (GT)₁₅ anchored CP (FIG. 27D, FIG. 27I) and averaged responses from 4 types of CPs (FIG. 27E, FIG. 27J). Pearson correlation coefficients and p-values are indicated.

FIG. 28: Normalized nanotube surface coverage change toward cDNA (FIGS. 28A-28E) and cRNA (FIGS. 28F-28J) against cytosine content of the target sequences. Responses of 5′ (CT)₁₅ anchored CP (FIG. 28A, FIG. 28F), 5′ (GT)₁₅ anchored CP (FIG. 28B, FIG. 28G), 3′ (CT)₁₅ anchored CP (FIG. 28C, FIG. 28H), 3′ (GT)₁₅ anchored CP (FIG. 28D, FIG. 28I) and averaged responses from 4 types of CPs (FIG. 28E, FIG. 28J). Pearson correlation coefficients and p-values are indicated.

FIG. 29: Sensor solvatochromic shift response toward cDNA (FIGS. 29A-29E) and cRNA (FIGS. 29F-29J) against guanine content of the target sequences. Responses of 5′ (CT)₁₅ anchored CP (FIG. 29A, FIG. 29F), 5′ (GT)₁₅ anchored CP (FIG. 29B, FIG. 29G), 3′ (CT)₁₅ anchored CP (FIG. 29C, FIG. 29H), 3′ (GT)₁₅ anchored CP (FIG. 29D, FIG. 29I) and averaged responses from 4 types of CPs (FIG. 29E, FIG. 29J). Pearson correlation coefficients and p-values are indicated

FIG. 30: Normalized nanotube surface coverage change toward cDNA (FIGS. 30A-30E) and cRNA (FIGS. 30F-30J) against guanine content of the target sequences. Responses of 5′ (CT)₁₅ anchored CP (FIG. 30A, FIG. 30F), 5′ (GT)₁₅ anchored CP (FIG. 30B, FIG. 30G), 3′ (CT)₁₅ anchored CP (FIG. 30C, FIG. 30H), 3′ (GT)₁₅ anchored CP (FIG. 30D, FIG. 30I) and averaged responses from 4 types of CPs (FIG. 30E, FIG. 30J). Pearson correlation coefficients and p-values are indicated.

FIG. 31: Influence of sensing conditions on sensor solvatochcromic responses and surface coverage change. (FIG. 31A-31C) N1-(GT)₁₅-SWCNT solvatochromic responses when bath sonication was carried out (FIG. 31A) after the 1-hour incubation at 37° C., (FIG. 31B) before the 1-hour incubation at 37° C., or (FIG. 31C) before the 1-hour incubation at room temperature. (FIG. 31D) N1-(GT)₁₅-SWCNT normalized surface coverage changes when bath sonication was carried out (left) after the 1-hour incubation at 37° C., (center) before the 1-hour incubation at 37° C., or (right) before the 1-hour incubation at room temperature.

### DETAILED DESCRIPTION OF THE INVENTION

Reference numbers in superscripts herein refer to the corresponding literature provided in the citation list provided infra; the references are incorporated by reference herein.

### Definitions

Unless otherwise stated, the following terms used in the specification and claims are defined for the purposes of this Application and have the following meaning:

As used herein, the singular forms “a,” “an,” and “the” are intended to include the plural forms as well, unless the context clearly indicates otherwise. It will be further understood that the terms “comprises” and/or “comprising,” and/or “including” when used in this specification, specify the presence of stated features, elements, and/or components, but do not preclude the presence or addition of one or more other features, elements, components, and/or groups thereof.

As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Reference herein to any numerical range (for example, a concentration range) expressly includes each numerical value (including fractional numbers and whole numbers) encompassed by that range, and each sub-range within that range, even where not expressly recited herein. For the sake of brevity, each possible numerical value or sub-range within a recited range may not be expressly disclosed, but is intended to be included as part of the disclosure herein even where not expressly disclosed. For example, but without limitation, reference herein to a range of 10-100 herein includes all whole numbers of and fractional numbers between the upper (100) and lower (10) limit of the range, inclusive of the upper and lower limit, such that, e.g. 20-80, 20-50, 40-80, 20, 40, 50, and 80 etc. are all among the ranges and values disclosed and encompassed herein.

The term “about,” as used herein when referring to a measurable value such as an amount of an agent of this disclosure, time, temperature, and the like, is meant to encompass variations of ±5%, ±1%, ±0.5%, or even ±0.1% of the specified amount.

It should also be understood that, unless clearly indicated to the contrary, in any methods claimed herein that include more than one step or act, the order of the steps or acts of the method is not necessarily limited to the order in which the steps or acts of the method are recited.

As used herein, an “analyte” is any molecule or combination of molecules in a sample, the presence and/or concentration of which is desirous to be measured. In particular, an analyte may comprise a nucleic acid.

As used herein, “nucleic acid” or “nucleic acid sequence” refer to a DNA or RNA sequence comprised of two or more nucleotides. The term nucleic acid includes sequences that include base analogues of DNA and RNA for example, but not limited to 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

As used herein “anchor” refers to a nucleic acid sequence, covalently bound at the 5′ or 3′ end of a complementary region, or at a suitable position other than one of the termini of the complementary region, where the sequence is not complementary to and/or does not hybridize to, the analyte of interest. When the anchor is covalently bound at the 5′ or 3′ end of a complementary regions, it is preferable bound through a standard nucleic acid linkage. As used herein in reference to an anchor, “not complementary to” means that no contiguous segment of the anchor that contains greater than 50% of the bases of the anchor is complementary to a portion of the analyte of interest.

A “complementary region” of a surface-adsorbed nucleic acid is a nucleic acid having a sequence that is complementary to and/or hybridizes to, the target region of an analyte of interest.

As used herein, a “sample” refers to any composition or mixture that contains an analyte or analytes, the presence and/or concentration of which is desirous to be measured. The sample may be a collection of fluids, cells, or tissues present within or isolated from a subject.

“Subject” as used herein means a mammal, including, but not limited to, murines, simians, humans, mammalian farm animals, mammalian sport animals, and mammalian pets. Preferably, the subject is human.

### SWCNT System

Using tailored SARS-CoV-2 complement sequences as proof of concept, the inventors have developed a novel system and related method for detecting an analyte in a sample, the system comprising SWCNTs and surface-adsorbed nucleic acids, wherein the surface-adsorbed nucleic acids comprise a complementary region and at least one anchor, wherein the complementary region comprises a nucleic acid sequence that is complementary to and hybridizes to a target region of the analyte and wherein the at least one anchor comprises a nucleic acid sequence that is not complementary to the target region of the analyte

As demonstrated in the examples disclosed herein, by measuring the kinetics of the hybridization process, the inventors calculated and compared the enthalpy of SWCNT CP hybridization to the solution phase hybridization, and by using a SWCNT diameter dependent solvatochromism model, they correlated observed photophysical changes to the CP changes and solvent exposed surface area following hybridization. The inventors further surprisingly found that PL response specificity can be improved through the inclusion of anchor sequences, with anchor absent SWCNT CPs showing no specificity. Anchor sequence and location configurations were derived and studied, resulting in multiple promising candidates for both DNA and RNA targets. Target sequence choice was shown to play a role in hybridization and signal transduction. The systems developed by the inventors were assessed in biologically relevant testing conditions, demonstrating the efficacy of a new generation of hybridization sensors enabled by the ssDNA-SWCNT platform.

Accordingly, in a first aspect, the present disclosure provides a system for detecting an analyte in a sample comprising semiconducting single-walled carbon nanotubes (SWCNTs) and surface-adsorbed nucleic acids, wherein the surface-adsorbed nucleic acids comprise a complementary region and at least one anchor, wherein the complementary region comprises a nucleic acid sequence that is complementary to and hybridizes to a target region of the analyte and wherein the at least one anchor comprises a nucleic acid sequence that is not complementary to the target region of the analyte. In certain embodiments, the surface-adsorbed nucleic acids are surface-adsorbed single-strand nucleic acids

It will be understood that reference herein to “an” analyte or “a” complementary region, etc. is not intended to be limiting and that the system disclosed herein may, in certain embodiments, be configured for the detection of one unique analyte molecule or it may be configured for the detection of a multiplicity of different analytes, i.e. it may be configured to detect one, two, three, four, etc. specific analytes in a sample. In such a case the system may comprise multiple different SWCNTs with surface-adsorbed nucleic acids, or SWCNTs having multiple different surface-adsorbed nucleic acids, where each different surface-adsorbed nucleic acid has a nucleic acid sequence that is complementary to and hybridizes to a target region of the each of the analytes to be detected.

The SWCNTs of the system may be of any chirality. In certain embodiments, the SWCNTs may have a diameter of greater than about 0.5 nm, greater than about 0.6 nm, greater than about, 0.7 nm, or greater than about 0.8 nm. In certain circumstances, the single walled carbon nanotube may have a diameter of less than about 2.0 nm, less than about 1.8 nm, less than about 1.6 nm, or greater than about 1.4 nm. For example, the single walled carbon nanotube can have a diameter of between about 0.8 nm and about 1.2 nm.

The surface-adsorbed nucleic acid comprises a complementary region and at least one anchor, wherein the complementary region comprises a nucleic acid sequence that is complementary to and/or hybridizes to a target region of the analyte. The surface-adsorbed nucleic acid may, in certain embodiments, be a surface-adsorbed single-strand nucleic acid, and may, in certain embodiments, be single strand DNA (ssDNA).

Anchor regions may be any sequence that is not complementary to the target region of the analyte. The anchor may be at the 5′ end of the complementary region, it may be at the 3′, or there may be an anchor at both the 5′ and 3′ ends of the complementary region. Anchors may also be present at sites within the surface-adsorbed nucleic acids other than the termini. The anchor regions may be of any length. In certain embodiments, the anchor region may be 6-80 nucleotides in length. In certain embodiments, the anchor may be 10-40 nucleotides in length, or it may be 30 nucleotides in length. In certain embodiments, the anchor may comprise repeating dimers, i.e. repeating CT, TC, AG, GA, CG, GC, AC, or CA dimers. In certain embodiments the anchor comprises repeating dimers of GT or CT, i.e. (GT)ₓ or (CT)ₓ, where x is 3-40. In certain embodiments, x may be 15.

The dissociation constant of the complementary region for the target region of the analyte in certain embodiments is about 5-20 nM. In particular embodiments, the dissociation constant may be about 11-13 nM, and in other embodiments, it may be about 11 nM for DNA, and about 13 nM for RNA.

The analyte can be any pathogen or other component in a sample whose detection is desired. In certain embodiments, the analyte may be a microbe, and in other embodiments, it may be a virus. The analyte may comprise a nucleic acid selected from the group consisting of single ssDNA, micro RNA, and viral RNA.

In certain embodiments, the analyte may be a SARS-CoV-2 virus, and in other embodiments, the target region of the analyte may be selected from the region encoding the spike protein, the region encoding the membrane protein, the region encoding the nucleocapsid protein, the region encoding the envelope protein, a region encoding a non-functional protein, or a non-coding region of a SARS-CoV-2 virus, and the surface-adsorbed nucleic acids may be complementary to a sequence selected from a SARS-CoV-2 virus, wherein the sequence is selected from the region encoding the spike protein, the region encoding the membrane protein, the region encoding the nucleocapsid protein, the region encoding the envelope protein, a region encoding a non-functional protein, or a non-coding region. In yet further embodiments, the target region of the analyte may be selected from SEQ ID Nos 1-11, and the surface-adsorbed nucleic acids may be complementary to a sequence selected from SEQ ID NOs 1-11.

The sample may be a biological sample, and in certain embodiments a biological sample collected from a human subject. The biological sample may be a biological fluid that may be present in, or withdrawn or otherwise extracted from, a subject or other biological source. Exemplary biological samples include, but are not limited to, saliva, blood, urine, tissue, cells, and nasopharyngeal swabs. Biological samples may also include serum and serosal fluids, plasma, lymph, cystic fluid, tear drops, feces, sputum, mucosal secretions of the secretory tissues and organs, vaginal secretions, ascites fluids, fluids of the pleural, pericardial, peritoneal, abdominal and other body cavities, and fluids collected by bronchial lavage and the like. Biological fluids may also include liquid solutions contacted with a subject or biological source, for example, culture medium.

### Methods of Detecting an Analyte in a Sample

In a second aspect, the present disclosure provides a method for detecting an analyte in a sample, the method comprising providing a system as described above in solution, combining the sample with the system, incubating the system and the sample, and measuring the photoluminescence of the system, wherein a shift in wavelength and/or intensity of the system relative to the system without exposure to analyte indicates the presence of the analyte.

It will be understood that in the methods disclosed herein, the system may be in any solution, e.g. in a buffer, in water, etc., that allows the surface-adsorbed nucleic acids of the system to hybridize to the target region of the analyte. That the system is in solution does not preclude, e.g., it being adhered to a solid surface (e.g. a sensor surface) provided that a liquid is added to the surface. In such a case, the liquid added to the surface may be the sample in solution, and when that is the case, it will be understood that “providing a system in solution” is intended to encompass the addition of a solution at the same time as when the sample is combined with the system.

The methods disclosed herein may also be used to determine the concentration of the analyte in the sample, by comparison of the shift in wavelength and/or intensiy of the PL of the system relative to standard calibration samples.

Hybridization of the analyte to the surface-adsorbed nucleic acids results in a shift in wavelength and/or intensity of the PL of the SWCNT system, which can be used to confirm presence of the analyte(s) of interest, as well as quantify the concentration of the analyte(s) when compared to the PL of the system absent exposure to the analyte(s) of interest. Suitable comparisons are within the purview of one of skill in the art.

The analyte may have a concentration in the sample of at least about 0.0001 nM, or a range of about 0.0001 nM to about 1000 nM. In certain embodiments, the concentration of the sample is about 500 nM. In certain embodiments, the analyte is detected at a concentration of less than about 0.001 nM, of less than about 0.01 nM, of less than about 0.1 nM, of less than about 1 nM, of less than about 10 nM, of less than about 100 nM, or of less than about 1000 nM. The analyte may also be present in the solution at least about 10² copies per mL

The system and sample may be incubated, i.e. allowed to sit, for a duration and at a temperature suitable for the particular analyte to be detected. In certain embodiments, the system and sample may be incubated for about 1 hour at about 37° C.

Measurement of the PL of the system following incubation is within the purview of one of skill in the art. In certain embodiments, the PL is measured in the 850-1250 nm range.

In the methods disclosed herein, the analyte can be any pathogen or other component in a sample whose detection is desired. In certain embodiments, the analyte may be a microbe, and in other embodiments, it may be a virus. The analyte may comprise a nucleic acid selected from the group consisting of single ssDNA, micro RNA, and viral RNA.

In certain embodiments, the analyte to be detected by the methods disclosed herein may be a SARS-CoV-2 virus, and in other embodiments, the target region of the analyte may be selected from the region encoding the spike protein, the region encoding the membrane protein, the region encoding the nucleocapsid protein, the region encoding the envelope protein, a region encoding a non-functional protein, or a non-coding region of a SARS-CoV-2 virus, and the surface-adsorbed nucleic acids may be complementary to a sequence selected from a SARS-CoV-2 virus, wherein the sequence is selected from the region encoding the spike protein, the region encoding the membrane protein, the region encoding the nucleocapsid protein, the region encoding the envelope protein, a region encoding a non-functional protein, or a non-coding region. In yet further embodiments, the target region of the analyte may be selected from SEQ ID Nos 1-11, and the surface-adsorbed nucleic acids may be complementary to a sequence selected from SEQ ID NOs 1-11.

The sample may be a biological sample as discussed, supra, and in certain embodiments a biological sample collected from a human subject.

### EXAMPLES

### Example 1: Materials and Methods

### Materials

Raw CoMoCAT SWCNTs enriched in (6,5) chirality were purchased from Sigma-Aldrich and used without further processing (Lot #MKCM1708). Single-strand DNA and RNA oligonucleotides were purchased from Integrated DNA Technologies. All other chemicals were purchased from Sigma Millipore.

### Target Viral RNA Sequence Design

Sequences were chosen intuitively with the aid of software tools. UNAFold Software was used, which is a common folding algorithm that predicts nucleic acid foldings, hybridizations, and melting profiles using energy-based methods and dynamic programming.⁵⁷ We identified regions longer than 18 nucleotides that are predicted to be exposed with minimal secondary structure formation based on the SARS-CoV-2 genome. We further BLAST searched them to show 100% identity with their designated gene for SARS-CoV-2 and specificity against OC43, 229E, NL63, MERS, and SARS1 coronaviruses as well as any gene in the human genome.

### Nanosensor Preparation and Characterization

1 mg of CoMoCAT SWCNT and 1 mg of ssDNA were mixed in 1 mL of 100 mM NaCl. The mixture was ultrasonicated with ⅛″ probe tip (Cole-Parmer) for 30 minutes at 44% amplitude in an ice bath. The sample was then centrifuged twice at 30300 g for 1-hour (Eppendorf Centrifuge 5430R). After each centrifugation, the top 80% of the suspension was collected while the remaining 20% was discarded to remove unsuspended bundles. The centrifuged sample was dialyzed against PBS with a 1 mL dialysis device of 300 kDa MWCO (Spectra-Por) overnight to remove free DNA. The concentration of the SWCNT suspension was determined using its absorbance at 632 nm (Agilent Technologies, Cary 5000) and extinction coefficient of 0.036 mg L⁻¹ cm⁻¹. The sample was stored at 4° C. fridge for further use.

### High Throughput Screening and nIR Photoluminescence Spectra Processing

High throughput screening of the nanosensor library against the viral nucleotides was performed using a customized nIR microscope, which consists of a Zeiss Axio Vision inverted microscope body with a 20× objective, coupled to an Acton SP2500 spectrometer and liquid nitrogen cooled InGaAs 1D detector (Princeton Instruments). Dialyzed SWCNT dispersion was diluted to 0.5 mg L⁻¹ and allowed to equilibrate overnight at room temperature before hybridization experiments. For screening, 50 μM stock solution of each target oligonucleotide in PBS was prepared. In a 96-well plate, 200 μL of SWCNT dispersion (0.5 mg L⁻¹) was added to 2 μL of 50 μM oligonucleotide solution. Addition of 2 μL of PBS was used as a negative control. The mixture was incubated for 1-hour at 37° C., after which the SWCNT fluorescence signal was monitored under laser excitation (785 nm, 317 mW, B&W Tek Inc.). The fluorescence spectra from three replicates were collected from 950 to 1250 nm. Following the acquisition, the spectra was processed in a custom MATLAB code which interpolates the spectra to locate the peak wavelength. (6,5), (7,5) and (9,4) chirality were assigned to the peaks circa 990 nm, 1045 nm and 1128 nm respectively. The (7,5) and (9,4) peak wavelength were obtained subtracting the influences of nearby chiralities. Specifically, the spectra shoulder present near the (7,5) and (9,4) peaks were cropped out by subtracting a fourth-order polynomial fit of the surround regions. Peak wavelength of each sensor-nucleotide pair were then compared to the sensor-PBS negative control to calculate the wavelength shift.

Hybridization Experiments with Surfactants, Biofluids and Bath Sonication

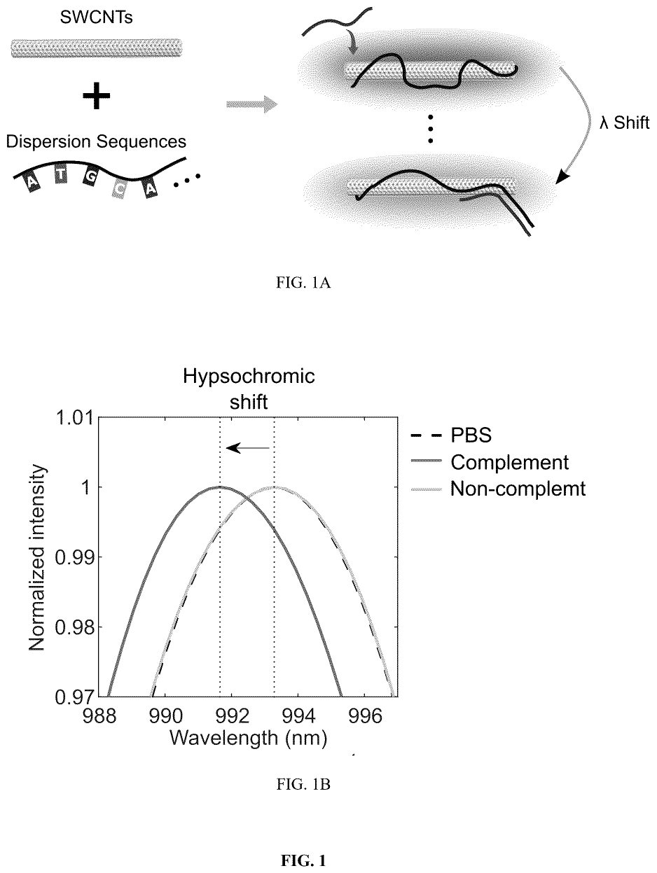

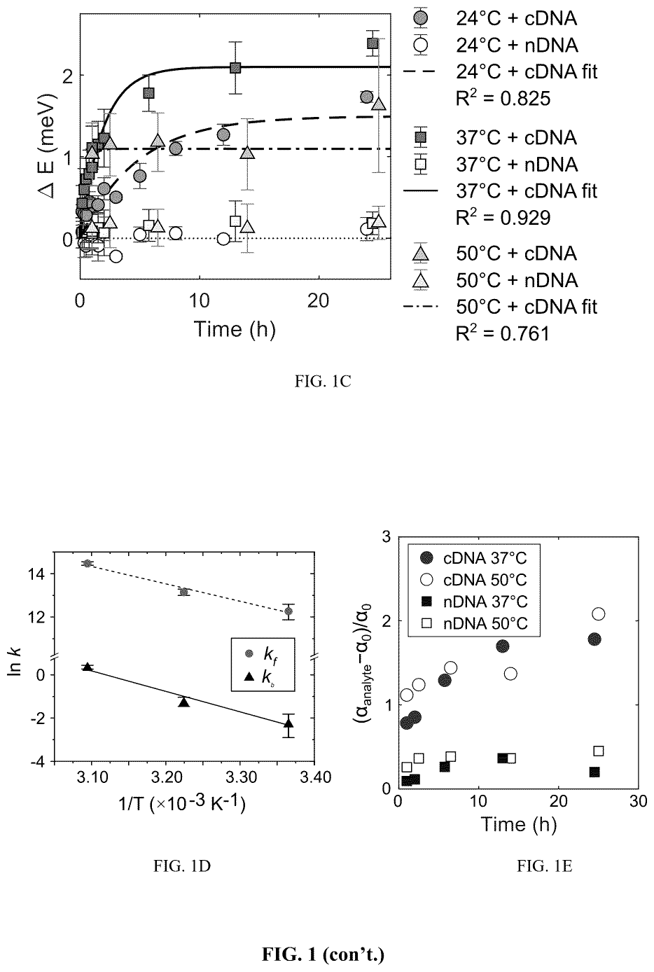

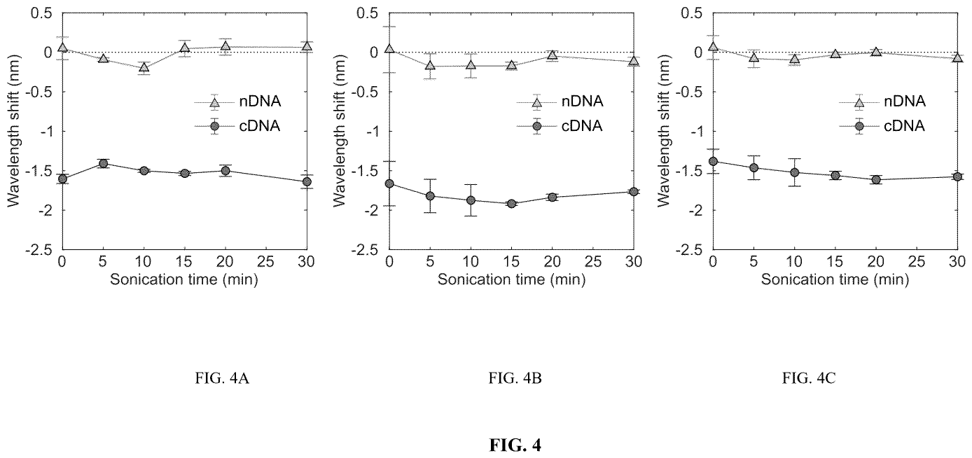

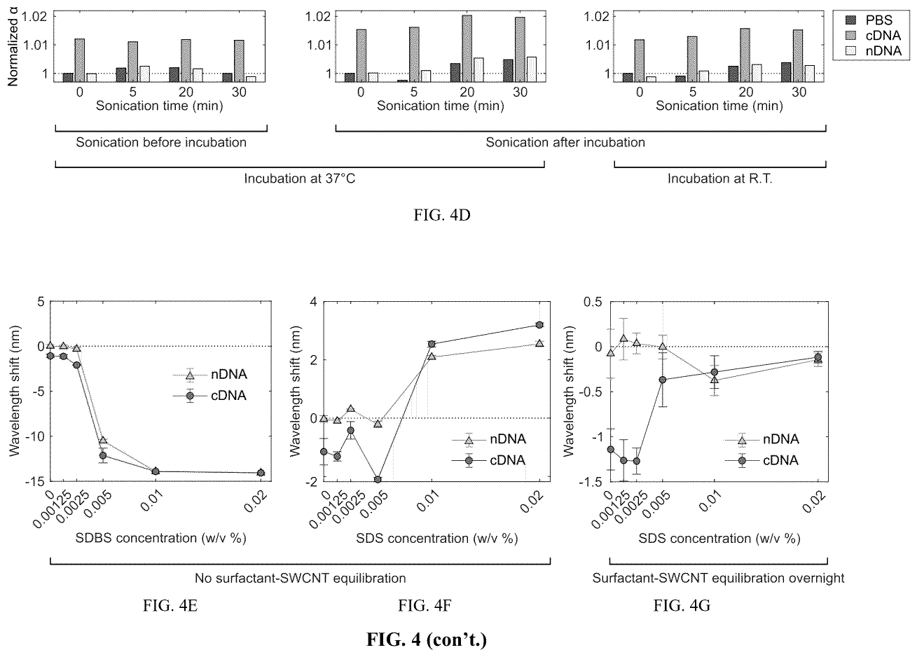

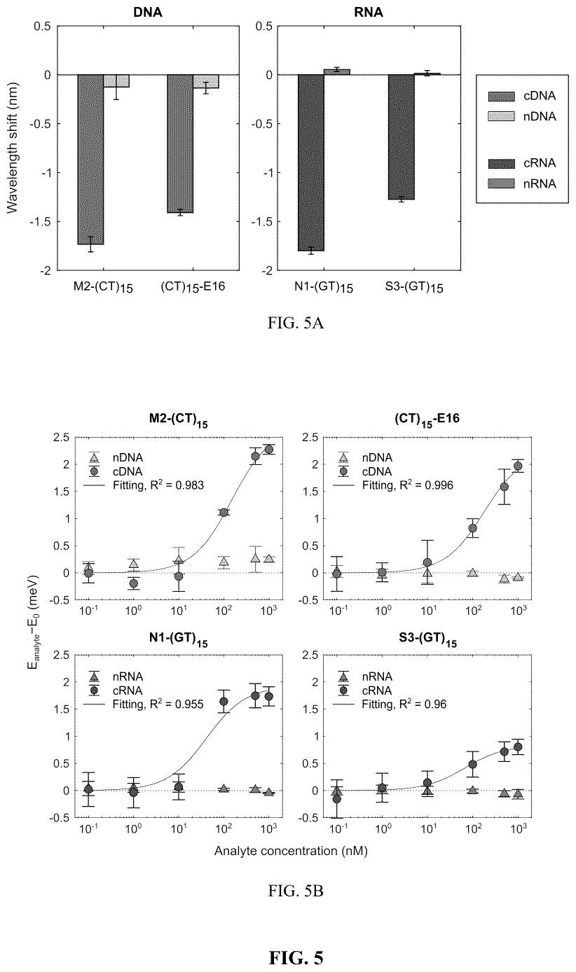

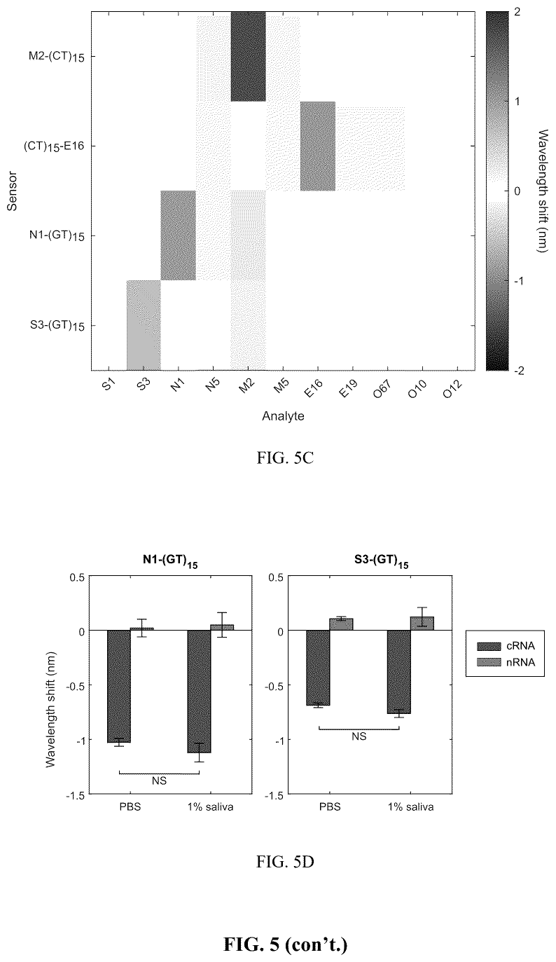

Hybridization experiments were conducted with 0.5 mg L⁻¹ SWCNT dispersion and target DNA or RNA at a final concentration of 500 nM, unless otherwise stated in the titration experiments in FIG. 5. Bath sonication was carried out using a 110 V ultrasonic bath (Arrayit Corporation). In these experiments, target DNA or RNA was first introduced to the SWCNT dispersion, followed by bath sonication then 1-hour incubation at specified temperature in FIG. 4 or 1-hour incubation then bath sonication. Hybridization experiments with surfactant were carried out at the final concentrations specified in FIG. 4. Surfactant solution was added to the SWCNT dispersions either at the same time as the addition of target DNA or 12-hour before the addition of target oligonucleotide. Regardless, the mixtures were incubated at 37° C. for 1-hour before its fluorescence spectra were acquired. Saliva sample was sourced from pooled human donors (MyBioSource) and was introduced to the SWCNT dispersion at a final concentration of 1% v/v. Spectra were acquired after 1-hour incubation at 37° C.

### Example 2: Results and Discussion

### Construct Synthesis, Design Study and Measurement Conditions

Nucleic acids are well known to form stable CPs around SWCNTs in aqueous environments via π-π stacking between the nucleoside and the SWCNT surface with the negatively charged phosphate backbones acting as the hydrophilic solution facing components.⁵⁸⁻⁶⁰ The adsorbed ssDNA on the SWCNT surface can bind to the complementary oligonucleotides introduced to the solution and result in a measurable wavelength modulation of the SWCNT PL. ssDNA-SWCNT constructs were synthesized using raw SWCNT material from the CoMoCAT process, enriched in the (6, 5) chirality species. A standard solution phase sonication method was used to disperse the SWCNTs against a specifically designed library of ssDNA oligonucleotides with complementary components to the 11 segments of the SARS-CoV-2 viral RNA genome (Table 1). Briefly, the ssDNA-SWCNT dispersions were created by ultrasonicating mixtures of ssDNA and CoMoCAT SWCNT in 100 mM NaCl solution, followed by centrifugation to remove SWCNT aggregates and dialysis with 1× phosphate buffered saline (PBS) buffer overnight to remove excess unbound ssDNA from solution. The concentration of the DNA-SWCNT dispersion was determined using the typical method of the absorbance at 632 nm and extinction coefficient of 0.036 L mg⁻¹ cm⁻¹.⁶¹

RNA secondary structures play a key role in affecting complementary hybridization. Predicting the secondary structure of RNA has long been studied to facilitate many genomics applications.^{62,63} In order to design sensors that can efficiently interact with the SARS-CoV-2 genes, we used a combination of software and manual alignment for identifying RNA genome regions that are predicted to be exposed regions of spike (S), nucleocapsid (N), membrane (M) and envelope (E) and open-reading frame (ORF) of SARS-CoV-2 genes. Some of the sequence design considerations include specificity to the target of interest and the minimization of secondary structures. The guanine-cytosine (GC) content of the sequences was also ensured to be evenly distributed to minimize hairpin and self-binding. Using this strategy 11 unique sequences for the S, N, M, and E genes and ORF of SARS-CoV-2 virus (Table 1) were selected.

TABLE 1
Target sequences from SARS-CoV-2 for detection with the ssDNA-SWCNT
construct

| | SEQ ID | | |
|---|---|---|---|
| Target | NO. | Sequences (5′→3′) | Gene encoding |
| S1712 | 1 | ACACTACTGATGCTGTCCGT | Spike (S) protein |
| S3555 | 2 | CCTCAATGAGGTTGCCAAGA | Spike (S) protein |
| N158 | 3 | TCACCGCTCTCACTCAACAT | Nucleocapsid (N) protein |
| N563 | 4 | CACGTAGTCGCAACAGTTCA | Nucleocapsid (N) protein |
| M296 | 5 | CTTTCAGACTGTTTGCGCGT | Membrane (M) protein |
| M566 | 6 | GTGACTCAGGTTTTGCTGCA | Membrane (M) protein |
| E160 | 7 | CCTTCTTTTTACGTTTACTCT | Envelope (E) protein |
| E198 | 8 | TTCTTCTAGAGTTCCTGATC | Envelope (E) protein |
| O1256 | 9 | AGTGTGCCTATTGGGTTCCA | Open reading frame (ORF) 1-ab |
| O6736 | 10 | TCAACCGCTGCTTTAGGTGT | Open reading frame (ORF) 1-ab |
| O10098 | 11 | TGTTCGCATTCAACCAGGAC | Open reading frame (ORF) 1-ab |

PBS was chosen to constitute a physiologically relevant but also buffered environment to reduce readout variability. Additional precautions were taken to improve measurement fidelity, including: diluting ssDNA-SWCNT to 0.5 mg/L to reduce aggregation, equilibrating SWCNT dilutions overnight, exciting the solution at low laser fluence (1.78×10⁴ mW/cm²), and well mixing during analyte incubation at consistent temperature.⁶⁴

For each hybridization experiment, DNA or RNA analyte was mixed with the SWCNT at a final concentration of 500 nM. Analytes can be complements to the ssDNA wrapping (cDNA, cRNA), randomly generated non-complementary control sequences (nDNA, nRNA), or PBS buffer. For clarity, ssDNA strands within the CP will be referred to as “adsorbed” and solution-phase testing strands referred to as “analyte”. The mixture was then incubated under a chosen experimental condition before the PL spectra was acquired in the 850-1250 nm range at approximately 2.56 pixel/nm using a high-throughput custom-made nIR fluorescence microscope. To extract SWCNT PL peak wavelength at sub-pixel resolutions, using the 990 nm peak as an example, a gaussian function was fit over data points near 990 nm to obtain the PL peak wavelength. The peak position after analyte incubation was compared to that of the PBS control to calculate wavelength shifts.

### Kinetics and Thermodynamics of SWCNT Surface Hybridization

In a typical experiment, we expected that the ssDNA-SWCNT PL peak would shift after coincubation with the complementary analyte while it would remain unchanged after coincubation with the non-complementary analyte (FIG. 1B). This observation is attributed to solvatochromism,⁵¹ where a shift in the optical transition energy, called the solvatochromic shift, results from a change in the exciton polarizability of SWCNT due to changes in its local dielectric environments (solvent Stark effect). The magnitude of this shift has been shown to scale approximately to the −4 power with SWCNT diameter (d⁻⁴) and with the square of the transition energy (Eᵢᵢ²).⁵¹ A hypsochromic shift, or increase in electronic transition energy, indicates a decrease in the effective dielectric constant of the SWCNT local environment. In this context, when the CP adsorbs more densely at the SWCNT surface, high dielectric constant water is excluded, causing the measured hypsochromic shift. In this way, a bathochromic shift (to the red or lower energy), conversely indicates a looser packing or lower density of the SWCNT corona.⁵³

For an adsorbed-analyte pair with a known response, we used the temporal changes of SWCNT PL (Table 2) at 24° C., 37° C. and 50° C. to first study the kinetics of the process. At all temperatures, we observed an increasing hypsochromic shift followed cDNA addition and negligible change followed nDNA addition (FIG. 1C). This selectivity suggests that hybridization plays a role. Nearly 20 hours and 10 hours were needed to reach steady state for 24° C. and 37° C., respectively and only 2 hours for 50° C. This slower kinetics for hybridization on SWCNT CP compared to the hybridization of free DNA in solution phase within minutes⁶⁵ indicates that there is polymer reorganization on the SWCNT surface. The response towards complementary analyte at 37° C. showed a higher steady state energy shift compared to 24° C. and 50° C. and less noise between triplicates compared to 50° C. We attribute this difference in noise as a reduced thermodynamic stability of the hybridized complexes, which had a denaturing temperature of 56.2° C.⁶⁶ This temperature induced denaturation increases distribution of SWCNTs between non-hybridized and hybridized states and as well as experimental variability. Due to the latter point, studies were subsequently carried out at 37° C. for 1 hour to for data reliability.

TABLE 2
Sequences of corona phase ssDNA and target used
in the kinetic studies

| Target | Sequences (5′→3′) |
|---|---|
| T1 | GGGGAACTTCTCCTGCTAGAAT |
| | (SEQ ID NO.: 12) |
| SWCNT CP | Sequences (5′→3′) |
| (CT)₁₅-T1 | CTCTCTCTCTCTCTCTCTCTCTCTCTCTCTATTCTA |
| | GCAGGAGAAGTTCCCC |
| | (SEQ ID NO.: 13) |

The bold-faced letters represent bases that form duplex.

To further characterize the hybridization kinetics, we used a Langmuir adsorption model assuming a reversible two-state hybridization without any intermediate:

$$cDNA ( A ) + Adsorbed ssDNA ( B ) ⇌ Hybridized duplex ( AB ) \qquad ( 1 )$$

The rate constants of the forward and backward reactions are denoted by k_{f} and k_{b}, and the equilibrium constant is denoted by K_{eq}=k_{f}/k_{b}. The rate of formation of the hybridized duplex can then be modeled using the following equation:

$$\frac{d {C}_{A B}}{dt} = {k}_{f} {C}_{A} {C}_{B} - {k}_{b} {C}_{A B} \qquad ( 2 )$$

where C_{A}, C_{B} and C_{AB} are the concentration of bulk cDNA in solution, the concentration of adsorbed ssDNA in SWCNT CP that is available for hybridization, and the concentration of hybridized duplex. All concentration units are normalized to number of moles per volume. t is time. According to the site balance, the total number of sites (C_{B,0}) can be calculated by summing the concentration of free sites (C_{B}) and the hybridized sites (C_{AB}). The rate of duplex formation can then be described by the following equation:

$$\frac{d {C}_{A B}}{dt} = {k}_{f} {C}_{A} ( {C}_{B , 0} - {C}_{A B} ) - {k}_{b} {C}_{A B} \qquad ( 3 )$$

The model further assumes that C_{A} is significantly higher than C_{B} such that C_{A} remains unchanged during the hybridization process. We assume that the SWCNT and its ssDNA corona has an approximate mass ratio of 1:1.⁶⁷ For a dispersion of 0.5 mg/L SWCNT, the adsorbed ssDNA concentration is approximately 32 nM, much lower than the analyte concentration of 500 nM. Thus, the analytical solution of equation 2 with initial condition of C_{AB}(0)=0 is:

$${C}_{A B} ( t ) = \frac{{C}_{A} {k}_{f}}{{k}_{b} + {C}_{A} {k}_{f}} {C}_{B , 0} ( 1 - {e}^{- ( {k}_{f} {C}_{A} + {k}_{b} ) t} ) \qquad ( 4 )$$

The normalized concentration of hybridized duplex can be correlated to the normalized energy shift

$$\frac{{C}_{A B}}{{c}_{AB , max}} = \frac{Δ E}{Δ {E}_{max}}$$

where C_{AB,max}=C_{B,0} is the maximum duplex concentration when hybridization reaches steady state.⁵³ Solving for the fluorescence energy shift we have Eqn. 5 to fit to our dataset.

$$Δ E ( t ) = \frac{{C}_{A} {k}_{f}}{{k}_{b} + {C}_{A} {k}_{f}} Δ {E}_{max} ( 1 - {e}^{- ( {k}_{f} {C}_{A} + {k}_{b} ) t} ) \qquad ( 5 )$$

The fitted models for 24° C., 37° C. and 50° C. are shown in FIG. 1C, with k_{f} of (2.18±0.75)×10⁵ M⁻¹ h⁻¹, (5.16±0.75)×10⁵ M⁻¹ h⁻¹ and (19.3±1.6)×10⁵ M⁻¹ h⁻¹ respectively, and k_{b} of 0.109±0.054 h⁻¹, 0.293±0.062 h⁻¹ and 1.42±0.12 h⁻¹ respectively. The hybridization rate constant k_{f} at 24° C. is similar to a previously reported value for a 22-mer DNA hybridization on SWCNT at room temperature modelled using only the forward hybridization reaction.⁵³ According to the Arrhenius law, the rate constants of hybridization (the forward reaction) and denaturation (the backward reaction) are each associated with an activation energy, which are E_{a,H} and E_{a,D} respectively. By constructing an Arrhenius plot (FIG. 1D), we found that E_{a,H} is 66.7 kJ mol⁻¹ and E_{a,D} is 78.6 kJ mol⁻¹. Under the two-state hybridization assumption which is usually valid for short oligonucleotides,⁶⁸⁻⁷⁰ the van't Hoff transition enthalpy of hybridization for this 22-mer DNA tested was calculated from

$$Δ {H}_{vH} = - R \frac{d ln {K}_{e q}}{d {T}^{- 1}}$$

to be −11.9 kJ mol⁻¹, which is more positive than that of a 21-mer DNA hybridizing with perfect match in solution (−707 kJ mol⁻¹)⁶⁵ and is rather on the scale of dimer duplex formation in solution (−44.4 to −30.1 kJ mol⁻¹).⁷¹ This indicates that DNA hybridization on SWCNT CP displays a smaller thermodynamic driving force compared to in solution, likely due to conformational constraints on SWCNT. Nevertheless, for a field effect transistor system composed of 10-mer ssDNA covalently attached to SWCNT the computed E_{a,H} and E_{a,D} were 142-202 kJ mol⁻¹ and 225-398 kJ mol⁻¹, respectively.²⁹ This suggests that the covalently attached DNA has greater energy barriers to hybridization, likely due to constraints on the number of available conformations.

To estimate the SWCNT corona-phase surface coverage, or solvent exposed surface area, we computed the SWCNT surface effective dielectric constant through the measured solvatochromic shifts and ratiometrically compared the results to a reference. A semi-empirical functional form of the SWCNT diameter solvatochromic shift was previously described.^{51,72}

$${( {E}_{ii} )}^{2} Δ {E}_{i i} = - L k [ \frac{2 ( ε - 1 )}{2 ε + 1} - \frac{2 ( {n}^{2} - 1 )}{{n}^{2} + 1} ] \frac{1}{{R}^{4}} = \frac{C}{{d}^{4}} \qquad ( 6 )$$

where Eᵢᵢ is the optical transition energy,

$$Δ {E}_{ii} = {E}_{ii} - {E}_{11}^{A i r}$$

is the difference between the optical transition energy in the dielectric environment (Eᵢᵢ) and the optical transition energy of pristine SWCNT in air

$$( {E}_{11}^{A i r} ) ,$$

L is a fluctuation factor, k is a scaling constant of the SWCNT polarizability, ε is the static dielectric constant, n is the refractive index, R is the nanotube radius and d is the nanotube diameter. The constant C gathers all the parameters that are constant for a specific chirality. In this work, the E₁₁ optical transitions of the (6,5), (7,5) and (9,4) SWCNTs were calculated via background fitting of the PL spectra. The optical transitions in air were then calculated according to:

$${E}_{11}^{A i r} = \frac{h c}{{A}_{1} + {A}_{2} d} + {A}_{3} \frac{cos θ}{{d}^{2}} \qquad ( 7 )$$

where h is Planck's constant, c is the speed of light, d is the SWCNT diameter, θ is the chiral angle corresponding to the SWCNT chirality (n, m), A₁=61.1 nm and A₂=1,113.6. By noting mod((n−m), 3)=j, A₃=−0.077 eV nm² for j=1 and A₃=0.032 eV nm² for j=2.

The proportionality constant C from Eqn. 6 was obtained by plotting (Eᵢᵢ)²ΔEᵢᵢ against 1/d⁴ for the different chiralities to calculate the slope from linear fitting. By comparing the constant to the slope of a reference system of SWCNT suspended in N-methyl-2-pyrrolidone (NMP), the effective dielectric constant, ε_{eff}, can be calculated by

$$\frac{C}{{C}_{N M P}} = \frac{\frac{{ε}_{eff} - 1}{2 {ε}_{eff} + 1} - \frac{{n}^{2} - 1}{2 {n}^{2} + 1}}{\frac{{ε}_{NMP} - 1}{2 {ε}_{N M P} + 1} - \frac{{n}_{N M P}^{2} - 1}{2 {n}_{N M P}^{2} + 1}} \qquad ( 8 )$$

where C_{NMP}=0.060 eV³ nm⁴, E_{NMP}=32.2, n_{NMP}=1.47 and n is the refractive index of DNA wrappings in water which is equal to that of water (n=1.333). Finally, assuming that the SWCNT surface coverage reflects a linear contribution from the solvent (water) and DNA wrappings to ε_{eff}. The relative surface coverage of SWCNT by its DNA wrapping, α, can be estimated by

$${ε}_{eff} = α {ε}_{D N A} + ( 1 - α ) {ε}_{Water} \qquad ( 9 )$$

where ε_{DNA} is the dielectric constant of the DNA wrappings (ε_{DNA} 4)⁷³ and ε_{Water} is the dielectric constant of water (ε_{Water}=88.1). Calculation of the surface coverage in this way is convenient and sufficient for this work. An alternative called the molecular probe adsorption (MPA) method would be to use the adsorption of fluorescent molecular probes for each of the experimental conditions.⁷⁴ The correlation between the solvatochromic surface coverage and MPA will be the topic of a future study, but we do not expect the difference to substantially change the conclusions of this current work.

To compare the results between experimental conditions, we calculate (α_{Analyte}−α₀)/α₀. The surface coverage upon PBS buffer addition, α₀, is considered as the initial DNA surface coverage without the introduction of solution phase analyte. A positive value means an increase in nanotube surface coverage by DNA resulting in a denser surface packing and negative values vice versa. As shown in FIG. 1E, SWCNT wrapping coverage increased upon complementary cDNA addition at both 37° C. and 50° C., while nDNA had no effect. Thus, hybridization resulted in a denser packing of the SWCNT surface while the nDNA was precluded.

### Adsorbed Strand Sequence Dependence on Hybridization Outcomes

While adsorbed, DNA conformation on the SWCNT surface likely interferes with the traditional geometries of nucleotide hybridization. Hybridization may occur on the SWCNT surface, in solution following partial detachment, or both. Previous work showed that an optimal “anchor sequence” may exist to assist with both SWCNT dispersion and presentation of the complementary strand for hybridization (FIG. 1A).⁵⁴ Anchor sequences, as the name suggests, were designed to adsorb to the SWCNT surface strongly such that the likelihood of complement regions desorbing to interact with solution phase analytes increases. In this configuration, it was argued that the anchor length is important to the accessibility of complement regions while being also not too far as to reduce solvatochromic effects.

To systematically understand the effects of the anchor segment, we created a test library of ssDNA-SWCNT comparing: 1) the presence of anchor, 2) the location of anchor at 5′ or 3′ end of the complementary region, 3) the length of anchor, and finally 4) the anchor nucleotide composition (Table 3, Table 4).

TABLE 3
Non-complimentary controls for sensing constructs.

| DNA | SEQ | | For DNA sensors |
|---|---|---|---|
| control | ID NO | DNA sequences (5′→3′) | targeting |
| nDNA 1 | 14 | GTTACATGTTCGTTGGGCTCTTC | All sequences |
| RNA | | | For RNA sensors |
| control | | RNA sequences (5′→3′) | targeting |
| nRNA 2a | 15 | ACACUACUGAUGCUGUCCGU | S3555, N158 |
| nRNA 2b | 16 | CCUCAAUGAGGUUGCCAAGA | S1712 |
| nRNA 2c | 17 | CACGUAGUCGCAACAGUUCA | M296 |
| nRNA 2d | 18 | CUUUCAGACUGUUUGCGCGU | N563 |
| nRNA 2e | 19 | GUGACUCAGGUUUUGCUGCA | E160 |
| nRNA 2f | 20 | CCUUCUUUUUACGUUUACUCU | M566 |
| nRNA 2g | 21 | UUCUUCUAGAGUUCCUGAUC | O6736 |
| nRNA 2h | 22 | AGUGUGCCUAUUGGGUUCCA | O10098 |
| nRNA 2i | 23 | UCAACCGCUGCUUUAGGUGU | E198 |
| nRNA 2j | 24 | UGUUCGCAUUCAACCAGGAC | O1256 |

The bold-faced letters represent bases that form duplex.

TABLE 4
Library of ssDNA sequences to form the SWCNT corona phase. Bold-faced letters
represent the complementary region.

| | SEQ ID | | |
|---|---|---|---|
| Name | NO | Sequences (5′→3′) | |
| S1 | 25 | ACGGACAGCATCAGTAGTGT | Complementary to S1712 |
| S3 | 26 | TCTTGGCAACCTCATTGAGG | Complementary to S3555 |
| N1 | 27 | ATGTTGAGTGAGAGCGGTGA | Complementary to N158 |
| N5 | 28 | TGAACTGTTGCGACTACGTG | Complementary to N563 |
| M2 | 29 | ACGCGCAAACAGTCTGAAAG | Complementary to M296 |
| M5 | 30 | TGCAGCAAAACCTGAGTCAC | Complementary to M566 |
| E16 | 31 | AGAGTAAACGTAAAAAGAAGG | Complementary to E160 |
| E19 | 32 | GATCAGGAACTCTAGAAGAA | Complementary to E198 |
| O10 | 33 | GTCCTGGTTGAATGCGAACA | Complementary to 010098 |
| O12 | 34 | TGGAACCCAATAGGCACACT | Complementary to 01256 |
| O67 | 35 | ACACCTAAAGCAGCGGTTGA | Complementary to 06736 |

| | | |
|---|---|---|
| (CT)₁₅-S1 | 36 | CTCTCTCTCTCTCTCTCTCTCTCTCTCTCTACGGACAGCATC |
| | | AGTAGTGT |
| (GT)₁₅-S1 | 37 | GTGTGTGTGTGTGTGTGTGTGTGTGTGTGTACGGACAGCAT |
| | | CAGTAGTGT |
| (AT)₁₅-S1 | 38 | ATATATATATATATATATATATATATATATACGGACAGCAT |
| | | CAGTAGTGT |
| (CT)₁₀-S1 | 39 | CTCTCTCTCTCTCTCTCTCTACGGACAGCATCAGTAGTGT |
| (CT)₂₀-S1 | 40 | CTCTCTCTCTCTCTCTCTCTCTCTCTCTCTCTCTCTCTCTACG |
| | | GACAGCATCAGTAGTGT |
| (GT)₁₀-S1 | 41 | GTGTGTGTGTGTGTGTGTGTACGGACAGCATCAGTAGTGT |
| (GT)₂₀-S1 | 42 | GTGTGTGTGTGTGTGTGTGTGTGTGTGTGTGTGTGTGTGTAC |
| | | GGACAGCATCAGTAGTGT |
| S1-(CT)₁₅ | 43 | ACGGACAGCATCAGTAGTGTCTCTCTCTCTCTCTCTCTCTC |
| | | TCTCTCTCT |
| S1-(GT)₁₅ | 44 | ACGGACAGCATCAGTAGTGTGTGTGTGTGTGTGTGTGTGT |
| | | GTGTGTGTGT |
| S1-(AT)₁₅ | 45 | ACGGACAGCATCAGTAGTGTATATATATATATATATATAT |
| | | ATATATATAT |
| (CT)₁₅-S3 | 46 | CTCTCTCTCTCTCTCTCTCTCTCTCTCTCTTCTTGGCAACCT |
| | | CATTGAGG |
| (GT)₁₅-S3 | 47 | GTGTGTGTGTGTGTGTGTGTGTGTGTGTGTTCTTGGCAACC |
| | | TCATTGAGG |
| S3-(CT)₁₅ | 48 | TCTTGGCAACCTCATTGAGGCTCTCTCTCTCTCTCTCTCTC |
| | | TCTCTCTCT |
| S3-(GT)₁₅ | 49 | TCTTGGCAACCTCATTGAGGGTGTGTGTGTGTGTGTGTGT |
| | | GTGTGTGTGT |
| (CT)₁₅-N1 | 50 | CTCTCTCTCTCTCTCTCTCTCTCTCTCTCTATGTTGAGTGAG |
| | | AGCGGTGA |
| (GT)₁₅-N1 | 51 | GTGTGTGTGTGTGTGTGTGTGTGTGTGTGTATGTTGAGTGA |
| | | GAGCGGTGA |
| N1-(CT)₁₅ | 52 | ATGTTGAGTGAGAGCGGTGACTCTCTCTCTCTCTCTCTCTC |
| | | TCTCTCTCT |
| N1-(GT)₁₅ | 53 | ATGTTGAGTGAGAGCGGTGAGTGTGTGTGTGTGTGTGTGT |
| | | GTGTGTGTGT |
| (CT)₁₅-N5 | 54 | CTCTCTCTCTCTCTCTCTCTCTCTCTCTCTTGAACTGTTGCG |
| | | ACTACGTG |
| (GT)₁₅-N5 | 55 | GTGTGTGTGTGTGTGTGTGTGTGTGTGTGTTGAACTGTTGC |
| | | GACTACGTG |
| N5-(CT)₁₅ | 56 | TGAACTGTTGCGACTACGTGCTCTCTCTCTCTCTCTCTCTC |
| | | TCTCTCTCT |
| N5-(GT)₁₅ | 57 | TGAACTGTTGCGACTACGTGGTGTGTGTGTGTGTGTGTGT |
| | | GTGTGTGTGT |
| (CT)₁₅-M2 | 58 | CTCTCTCTCTCTCTCTCTCTCTCTCTCTCTACGCGCAAACAG |
| | | TCTGAAAG |
| (GT)₁₅-M2 | 59 | GTGTGTGTGTGTGTGTGTGTGTGTGTGTGTACGCGCAAACA |
| | | GTCTGAAAG |
| M2-(CT)15 | 60 | ACGCGCAAACAGTCTGAAAGCTCTCTCTCTCTCTCTCTCTC |
| | | TCTCTCTCT |
| M2-(GT)15 | 61 | ACGCGCAAACAGTCTGAAAGGTGTGTGTGTGTGTGTGTGT |
| | | GTGTGTGTGT |
| (CT)₁₅-M5 | 62 | CTCTCTCTCTCTCTCTCTCTCTCTCTCTCTTGCAGCAAAACC |
| | | TGAGTCAC |
| (GT)₁₅-M5 | 63 | GTGTGTGTGTGTGTGTGTGTGTGTGTGTGTTGCAGCAAAAC |
| | | CTGAGTCAC |
| M5-(CT)₁₅ | 64 | TGCAGCAAAACCTGAGTCACCTCTCTCTCTCTCTCTCTCTC |
| | | TCTCTCTCT |
| M5-(GT)₁₅ | 65 | TGCAGCAAAACCTGAGTCACGTGTGTGTGTGTGTGTGTGT |
| | | GTGTGTGTGT |
| (CT)₁₅-E16 | 66 | CTCTCTCTCTCTCTCTCTCTCTCTCTCTCTAGAGTAAACGTA |
| | | AAAAGAAGG |
| (GT)₁₅-E16 | 67 | GTGTGTGTGTGTGTGTGTGTGTGTGTGTGTAGAGTAAACGT |
| | | AAAAAGAAGG |
| E16-(CT)₁₅ | 68 | AGAGTAAACGTAAAAAGAAGGCTCTCTCTCTCTCTCTCTCT |
| | | CTCTCTCTCT |
| E16-(GT)₁₅ | 69 | AGAGTAAACGTAAAAAGAAGGGTGTGTGTGTGTGTGTGTG |
| | | TGTGTGTGTGT |
| (CT)₁₅-E19 | 70 | CTCTCTCTCTCTCTCTCTCTCTCTCTCTCTGATCAGGAACTC |
| | | TAGAAGAA |
| (GT)₁₅-E19 | 71 | GTGTGTGTGTGTGTGTGTGTGTGTGTGTGTGATCAGGAACT |
| | | CTAGAAGAA |
| E19-(CT)₁₅ | 72 | GATCAGGAACTCTAGAAGAACTCTCTCTCTCTCTCTCTCTC |
| | | TCTCTCTCT |
| E19-(GT)₁₅ | 73 | GATCAGGAACTCTAGAAGAAGTGTGTGTGTGTGTGTGTGT |
| | | GTGTGTGTGT |
| (CT)₁₅-O10 | 74 | CTCTCTCTCTCTCTCTCTCTCTCTCTCTCTGTCCTGGTTGAA |
| | | TGCGAACA |
| (GT)₁₅-O10 | 75 | GTGTGTGTGTGTGTGTGTGTGTGTGTGTGTGTCCTGGTTGA |
| | | ATGCGAACA |
| O10-(CT)₁₅ | 76 | GTCCTGGTTGAATGCGAACACTCTCTCTCTCTCTCTCTCTC |
| | | TCTCTCTCT |
| O10-(GT)₁₅ | 77 | GTCCTGGTTGAATGCGAACAGTGTGTGTGTGTGTGTGTGT |
| | | GTGTGTGTGT |
| (CT)₁₅-O12 | 78 | CTCTCTCTCTCTCTCTCTCTCTCTCTCTCTTGGAACCCAATA |
| | | GGCACACT |
| (GT)₁₅-O12 | 79 | GTGTGTGTGTGTGTGTGTGTGTGTGTGTGTTGGAACCCAAT |
| | | AGGCACACT |
| O12-(CT)₁₅ | 80 | TGGAACCCAATAGGCACACTCTCTCTCTCTCTCTCTCTCTC |
| | | TCTCTCTCT |
| O12-(GT)₁₅ | 81 | TGGAACCCAATAGGCACACTGTGTGTGTGTGTGTGTGTGT |
| | | GTGTGTGTGT |
| (CT)₁₅-O67 | 82 | CTCTCTCTCTCTCTCTCTCTCTCTCTCTCTACACCTAAAGCA |
| | | GCGGTTGA |
| (GT)₁₅-O67 | 83 | GTGTGTGTGTGTGTGTGTGTGTGTGTGTGTACACCTAAAGC |
| | | AGCGGTTGA |
| O67-(CT)₁₅ | 84 | ACACCTAAAGCAGCGGTTGACTCTCTCTCTCTCTCTCTCTC |
| | | TCTCTCTCT |

The bold-faced letters represent bases that form duplex.

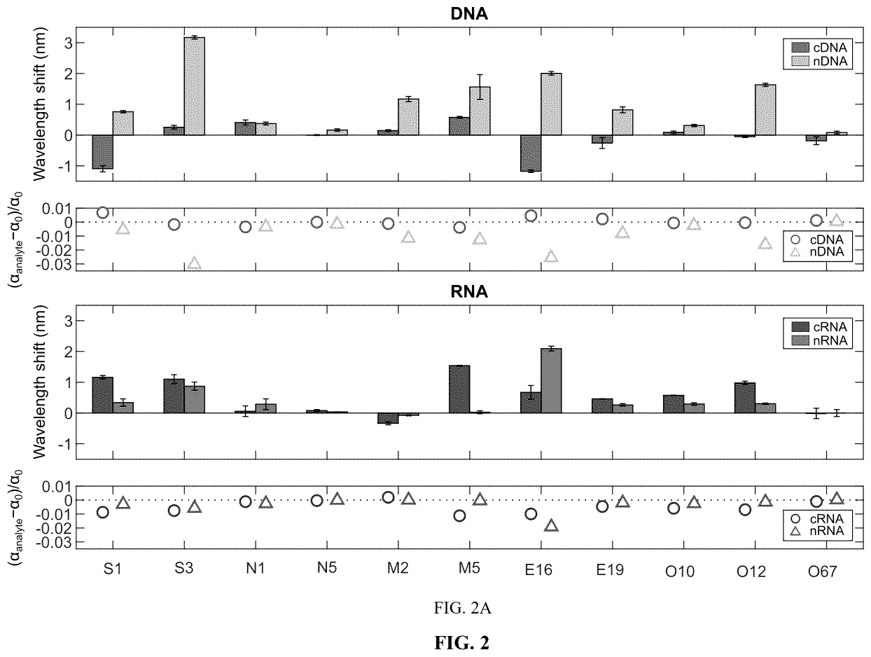

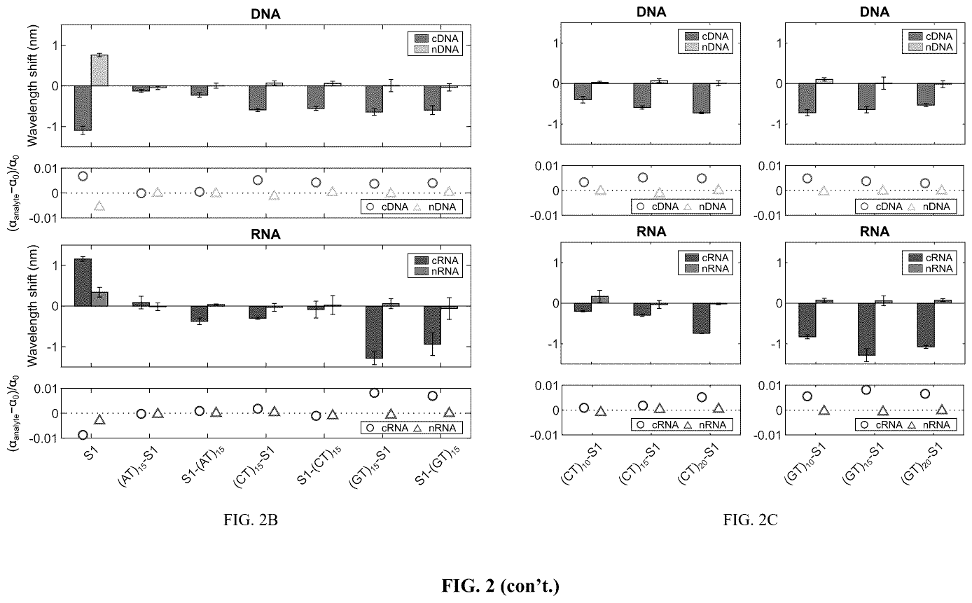

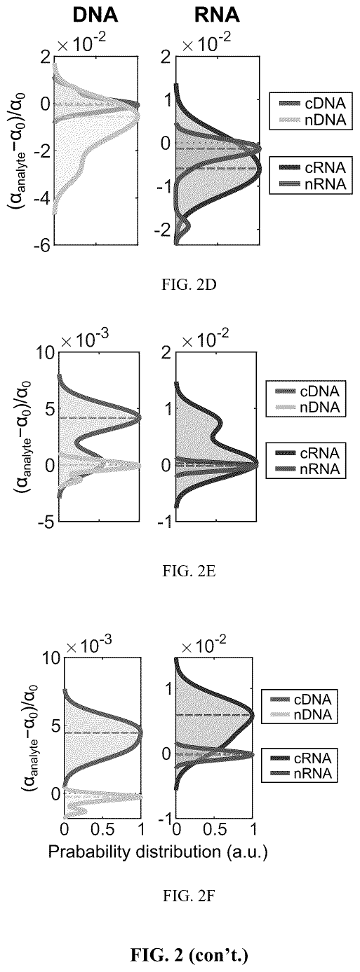

As a control we first tested SWCNT CPs without anchor segments. We found that the complementary analytes (cDNA and cRNA) gave inconsistent responses while the non-complementary analytes (nDNA and nRNA) resulted in mostly bathochromic shifts and looser DNA surface coverage (FIG. 2A). To better represent the results, we use the data set as a group to compare the probability distribution of the relative surface coverage changes following complement or random analyte addition (FIG. 2D). These results graphically showed that the two distributions were unimodal and overlapping for both DNA and RNA.

To study the effect of introducing anchor segments, we used the S1 sequence as an example. We attached anchor segments of (AT)₁₅, (CT)₁₅, or (GT)₁₅ to either the 3′ or 5′ end of S1. Results from FIG. 2B and FIG. 2E show that the inclusion of a 30-mer anchor imparted recognition specificity as shown by the hypsochromic shifts (increased surface coverage) in most experimental conditions, with the exception of (AT)₁₅ anchors cRNA. We hypothesized that the anchor regions improve surface adsorption of the CP, helping to specifically recruit complementary duplexes. According to the binding energy per nucleotide on SWCNT determined by Iliafar et al.,⁷⁵ the 30-mer (AT)₁₅, (CT)₁₅, or (GT)₁₅ anchors correspond to an additional 921, 606 and 858 k_{B}T in binding energy, much greater than that of the S1 complementary region alone (594 k_{B}T). Comparing across the different anchor compositions, (CT)₁₅ and (GT)₁₅ exhibited greater selective hypsochromic shift and greater surface coverage than (AT)₁₅ (FIG. 2B). We attribute this difference to the likely self-hybridization effects of (AT)₁₅ between CP anchor regions, reducing corona stability and presentation of the complementary region. Next, the 3′ or 5′ location of anchors did not show a significant difference (FIG. 2B). Finally, we assessed the responses from sensors with anchors of different lengths (FIG. 2C). We found that longer (CT)ₓ anchor lengths (x=10, 15, and 20) caused increased hybridization effects. While no such trend was found for the (GT)ₓ anchors, its overall response was higher (FIG. 2C). Given that guanine has a higher binding energy than cytosine on the SWCNT surface in aqueous phase,⁷¹⁷⁸ (CT)ₓ anchors partially makes up for the difference via length. Nonetheless, the inclusion of (CT)ₓ and (GT)ₓ anchor of any length led to hybridization specificity as clearly shown by the distinctly shifted probability distributions in FIG. 2F. Given these anchor study results, we designed a

### Screening Results Against Target Sequences

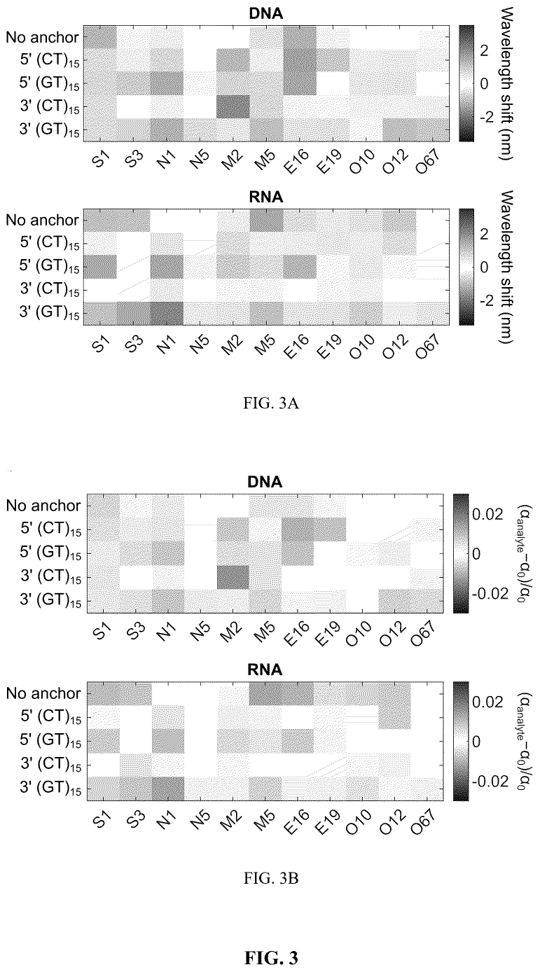

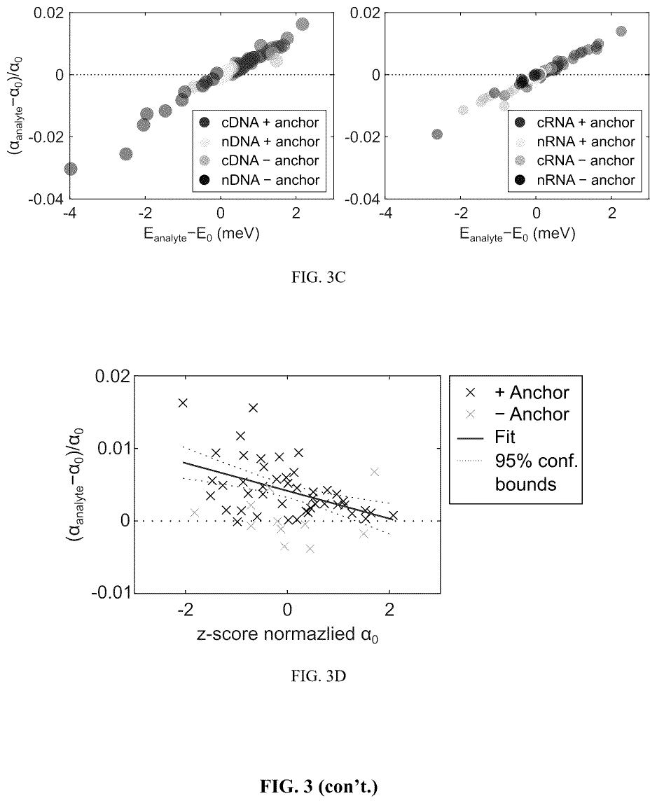

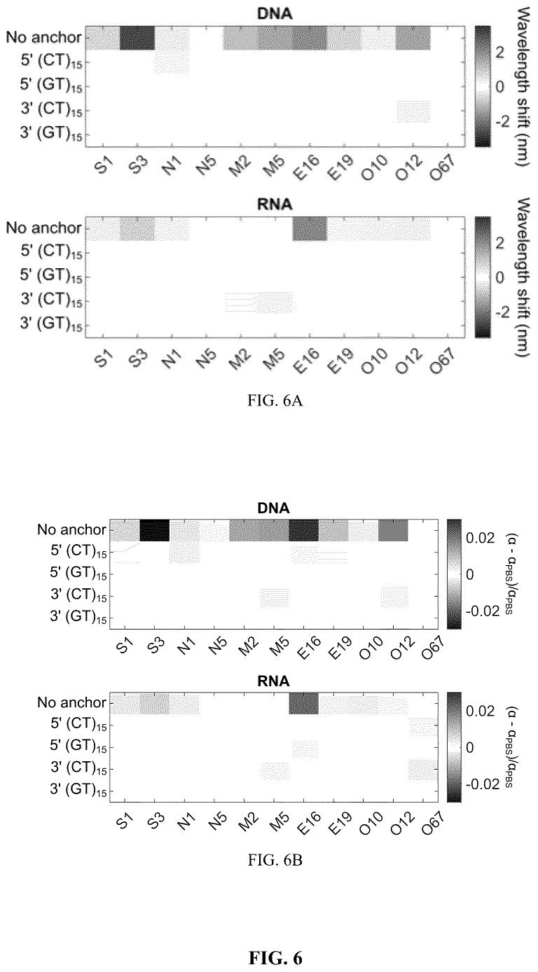

A library of ssDNA was created to suspend SWCNTs to target each of the aforementioned analyte sequences. Each ssDNA was composed of (CT)₁₅ or (GT)₁₅ anchor adjacent to either the 5′ or 3′ end of the complementary segment. Results showed that most of the constructs with anchors showed significant hypsochromic shifts in PL spectra toward cDNA and cRNA compared to the bathochromic shifts without anchors (FIG. 3A). Successful hybridization for constructs with anchors were further confirmed by the denser DNA wrapping on nanotube surface upon cDNA and cRNA introduction as shown in FIG. 3B. Negligible responses were shown for random controls (FIG. 6A, FIG. 6B). In a scatter plot of PL emission energy shift versus surface coverage change, we showed that the larger and predictable responses came from presence of anchors and complement sequences (FIG. 3C).

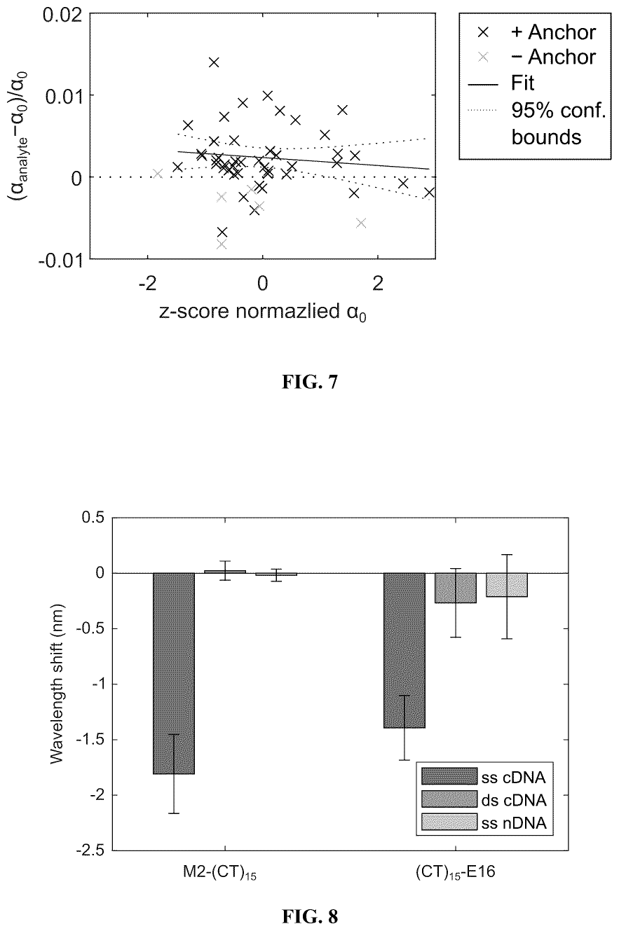

Looking at the data set as a whole, we found a linear correlation between the initial SWCNT surface coverage and its hybridization response (FIG. 3D). The negative correlation indicates that a loosely packed CP could accommodate more complementary targets for hybridization. This was not seen in non-anchored results (FIG. 7), suggesting that the anchor plays a major role in analyte recruitment.

We statistically investigated the impacts of anchor composition, (CT)₁₅ or (GT)₁₅, and location, 5′ or 3′, on PL responses as shown in Table 5. For detecting RNA targets only, constructs with (GT)₁₅ anchor regardless of attachment location demonstrated greater hypsochromic shifts and greater increase in surface coverage upon hybridization compared to those with (CT)₁₅ anchor. This could be attributed to the stronger adsorption strength of (GT)₁₅ anchor (858 k_{B}T) on SWCNT surface than (CT)₁₅ anchor (606 k_{B}T), leading to stronger adsorption stability for hybridized duplex. Anchor location did not show a clear trend.

TABLE 5
The p-values of paired, two-tailed non-parametric Wilcoxon
signed rank tests with significance level of 0.05 on the
impacts of anchor composition and location on PL responses.

| | | DNA analytes | | RNA analytes | |
|---|---|---|---|---|---|
| | | | Normalized | | Normalized |
| | | Solvatochromic | surface coverage | Solvatochromic | surface coverage |
| | | shift | change | shift | change |
| (CT)₁₅ | 5′ | P = 0.779 | P = 0.765 | P = 0.0186 | P = 0.0244 |
| vs. | 3′ | P = 0.083 | P = 0.102 | P < 0.001 | P < 0.001 |
| (GT)₁₅ | | | | | |
| 5′ vs. | (CT)-₁₅ | P = 0.175 | P = 0.24 | P = 0.042 | P = 0.083 |
| 3′ | (GT)-₁₅ | P = 0.577 | P = 0.52 | P = 0.206 | P = 0.206 |

As a separate control, we tested prehybridized double stranded DNA (dsDNA) as analytes on our best DNA-performing constructs (M2-(CT)₁₅, (CT)₁₅-E16) (FIG. 8). All dsDNA experimental conditions, non-complement or complement, produced negligible PL responses. This is consistent with our thermodynamic estimates that solution phase canonical hybridization is favored over its SWCNT CP counterpart. As a result, we further hypothesized that secondary structures formation within analyte strands (e.g. hairpins and self-dimers), although designed to be minimal by prediction, would be unavoidable in reality and similarly contribute to attenuated PL responses.

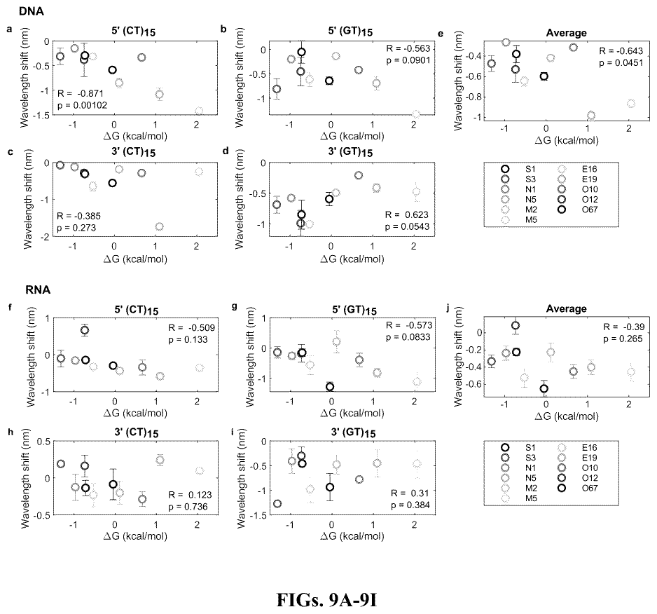

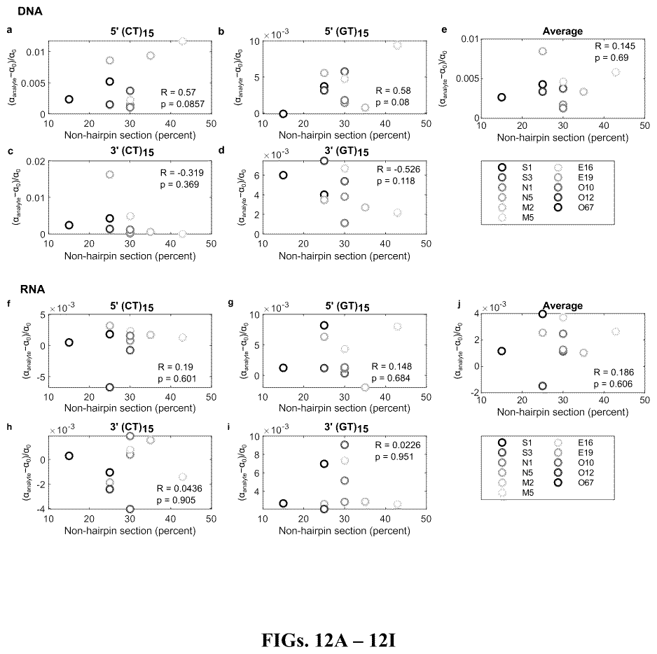

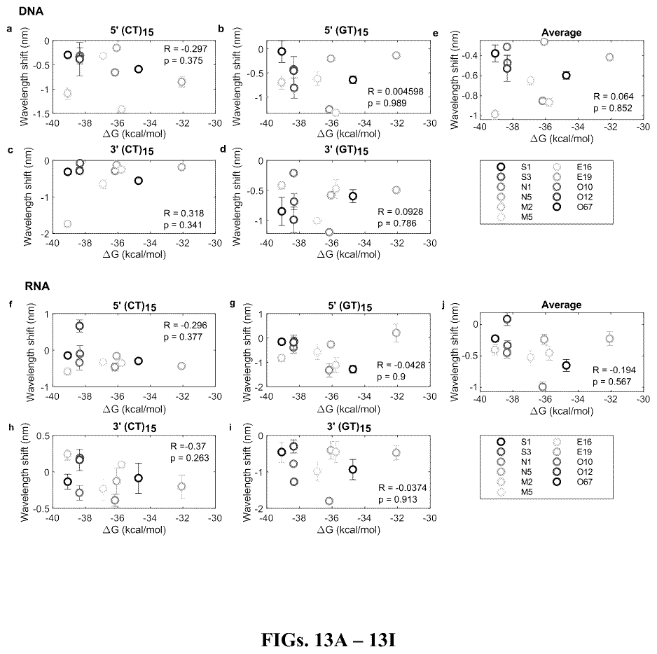

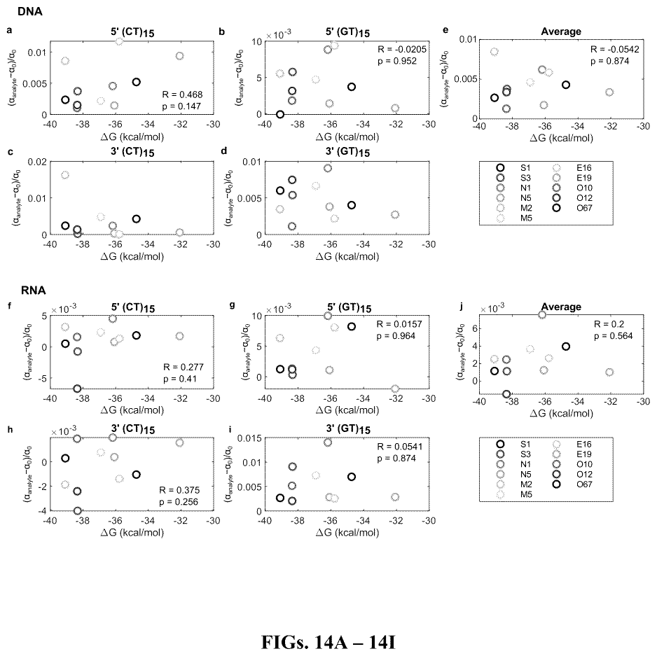

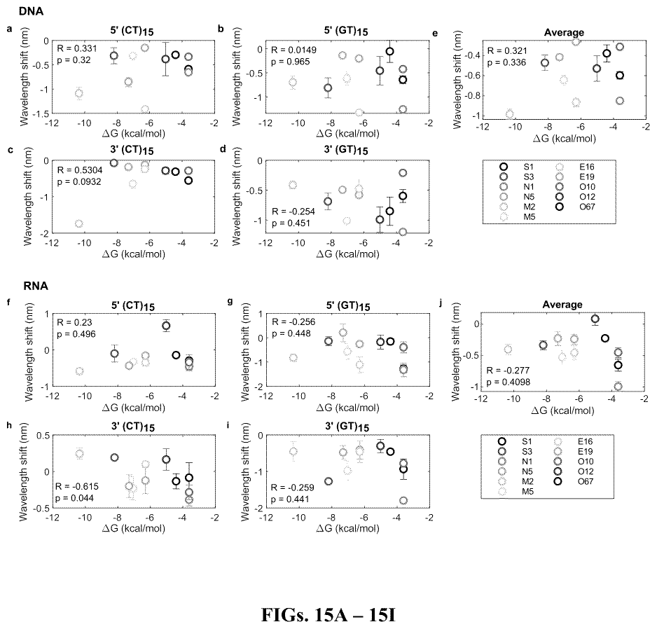

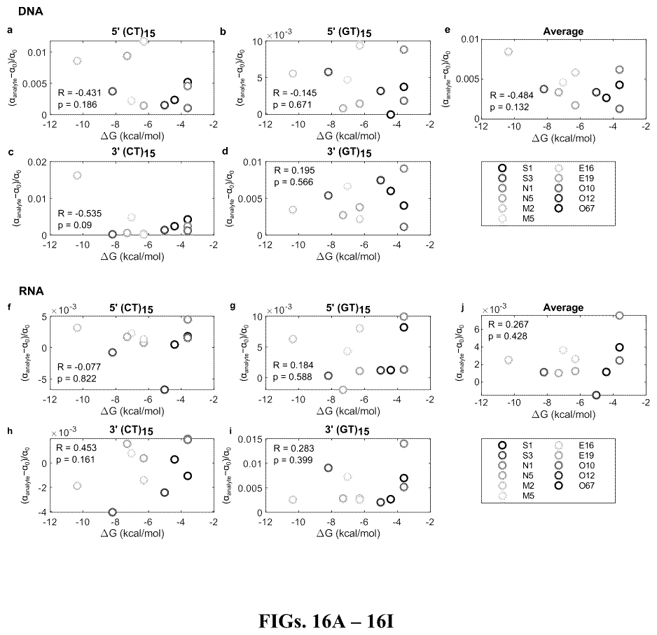

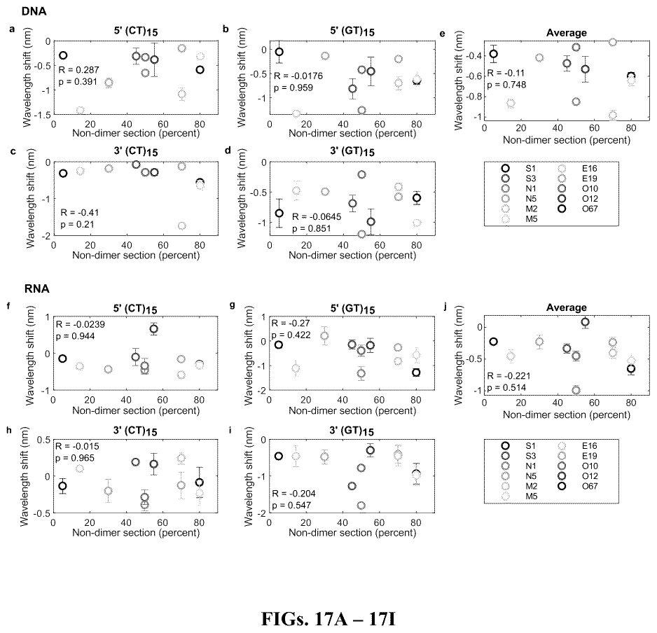

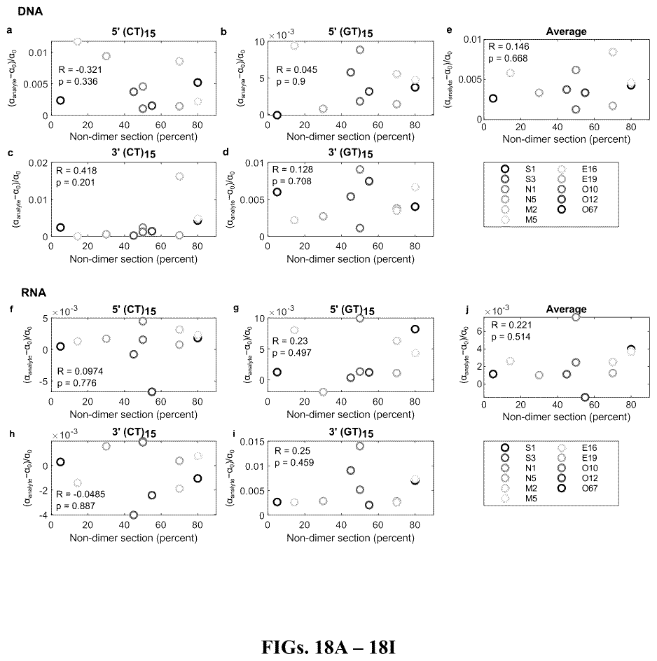

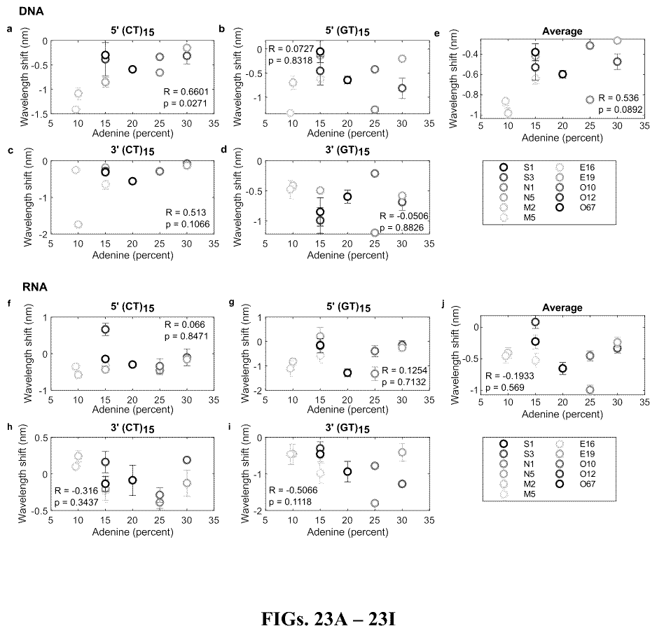

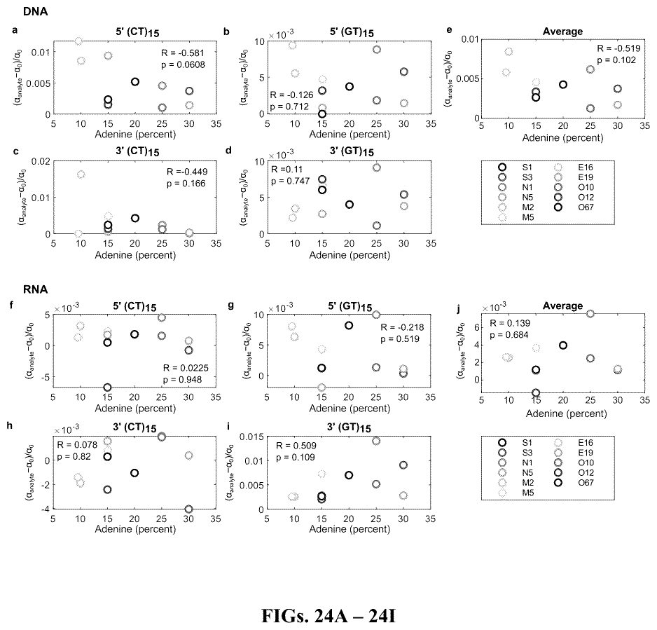

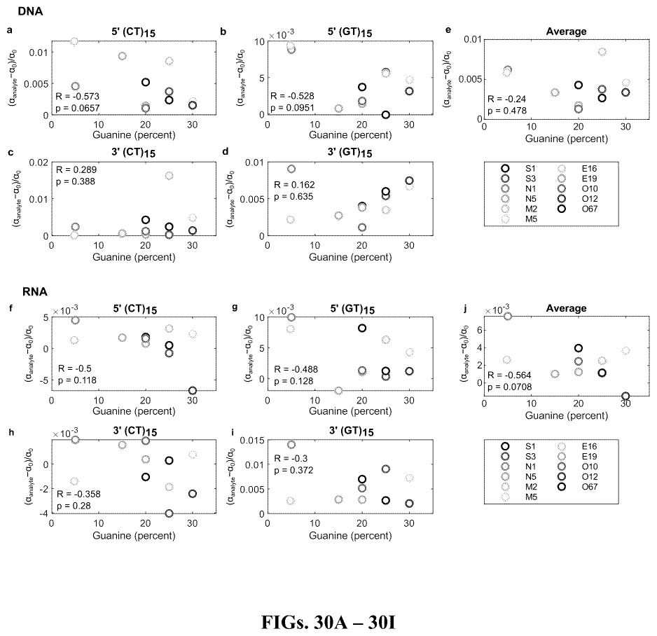

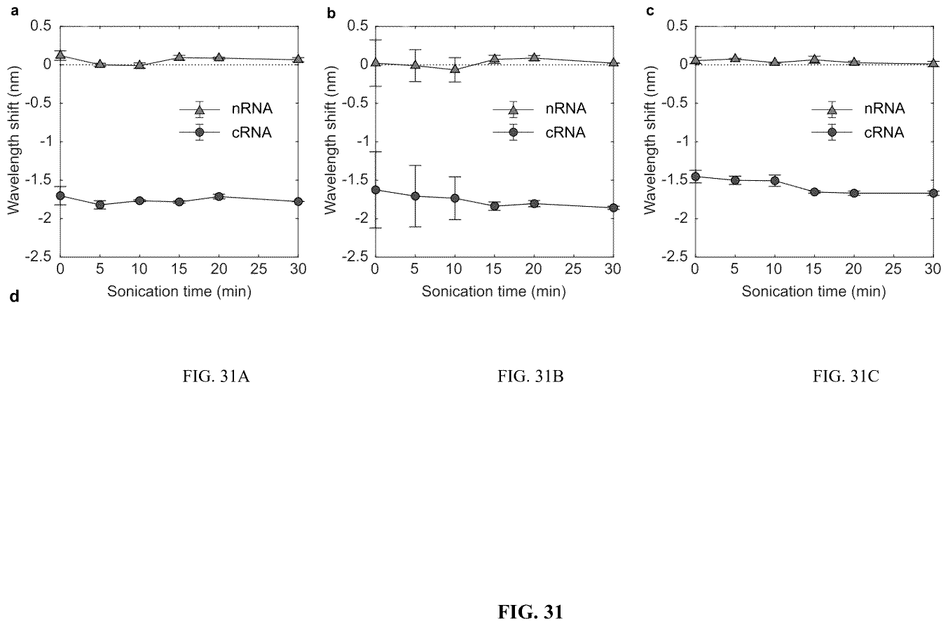

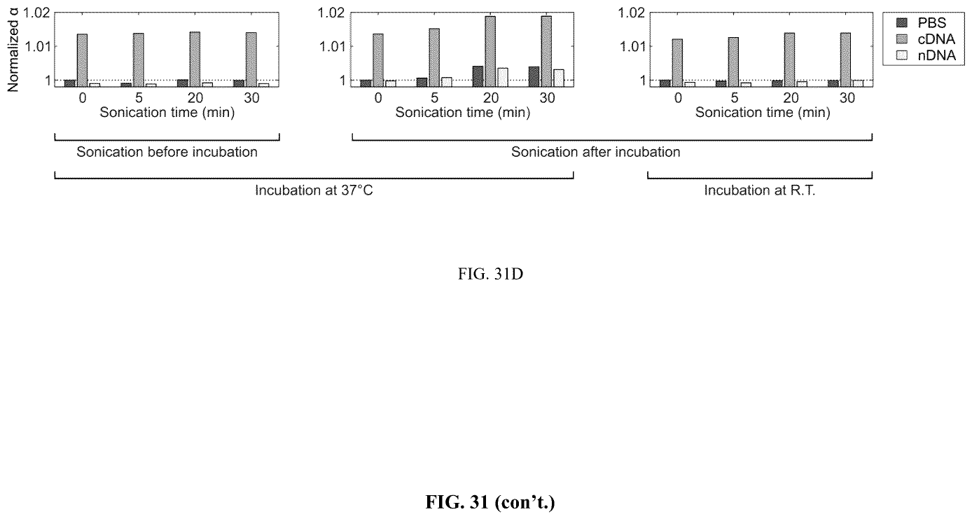

To test the above hypothesis and study analyte sequence property dependence, we used the following metrics as features of the analyte sequences (FIGS. 9-30). We correlated these features to our sensor responses to gain an understanding of which features influence the hybridization events. Further description of each metric can be found below.
1. Most negative free energy of hairpin formation among all potential hairpins, ΔG_{Hp}
2. % length of the shortest single-stranded section among all hairpins, L_{Hp}
3. Free energy of hybridization, ΔG_{Hyb}
4. Most negative free energy of self-dimerization among all potential self-dimers, ΔG_{Dm}
5. % length of the single-stranded section of the self-dimer in (4), L_{Dm}
6. Minimum of (2) and (5), L_{Min}
7. Number of unique self-dimers possible, N_{Dm}
8. A, T, G, C content individually

We calculated the correlation coefficients between these analyte properties and SWCNT PL responses, grouped by anchor type (5′/3′ and (CT)₁₅/(GT)₁₅). Statistically significant correlations were shown in Table 6.

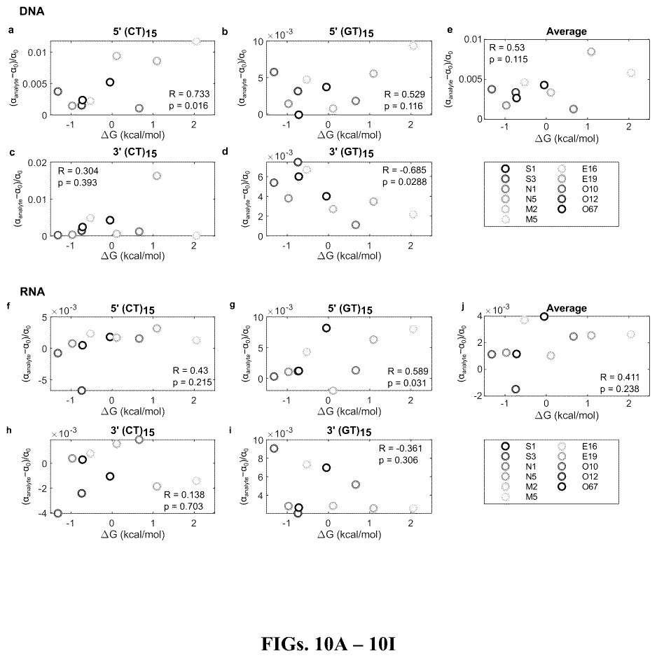

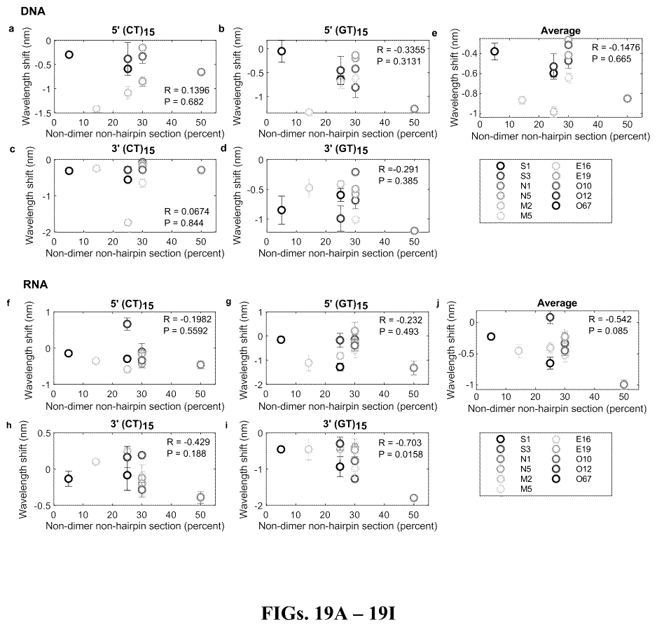

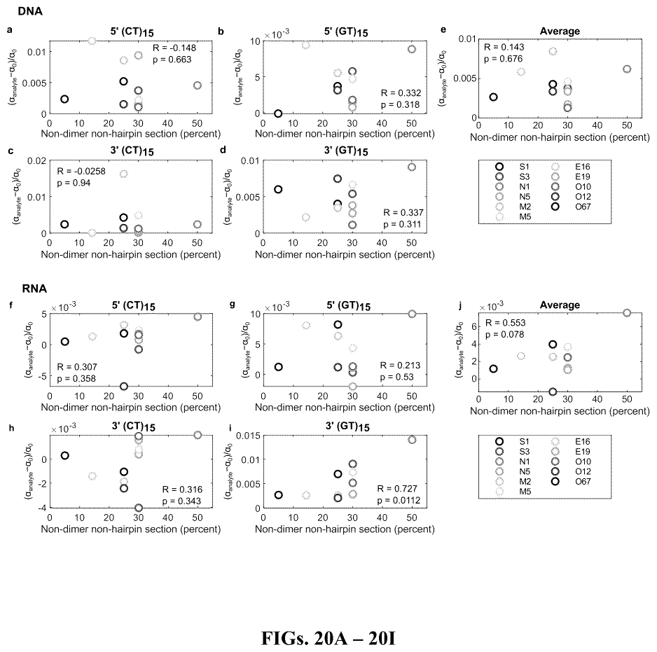

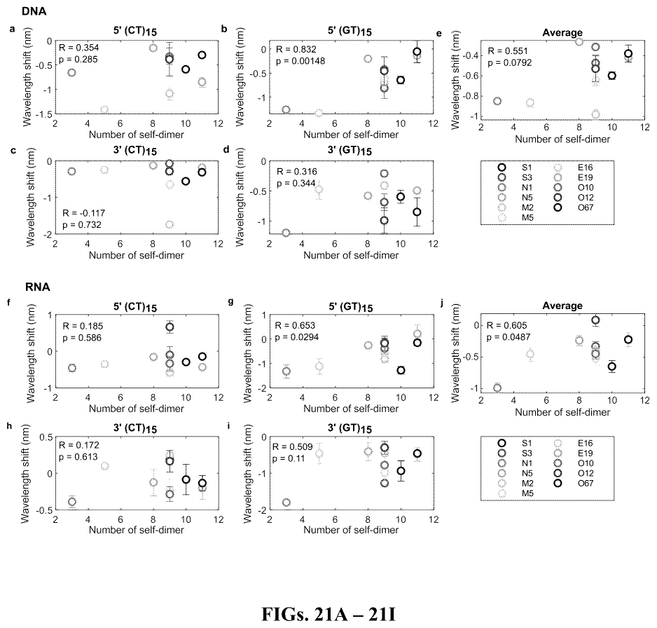

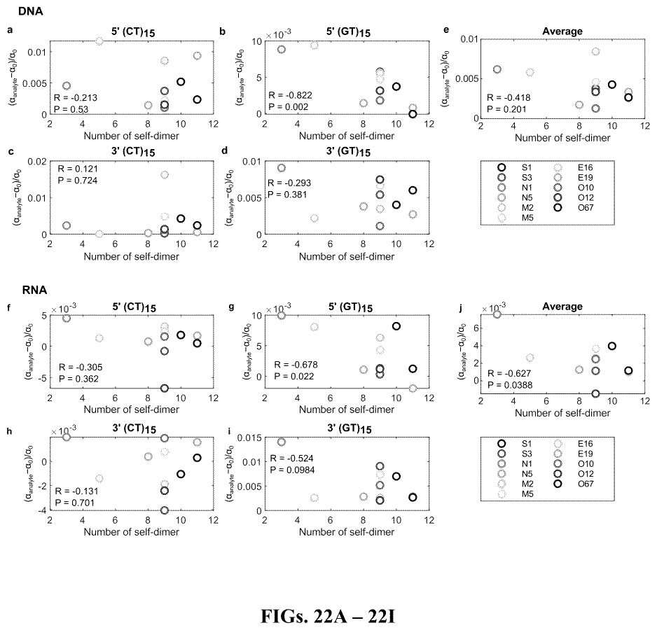

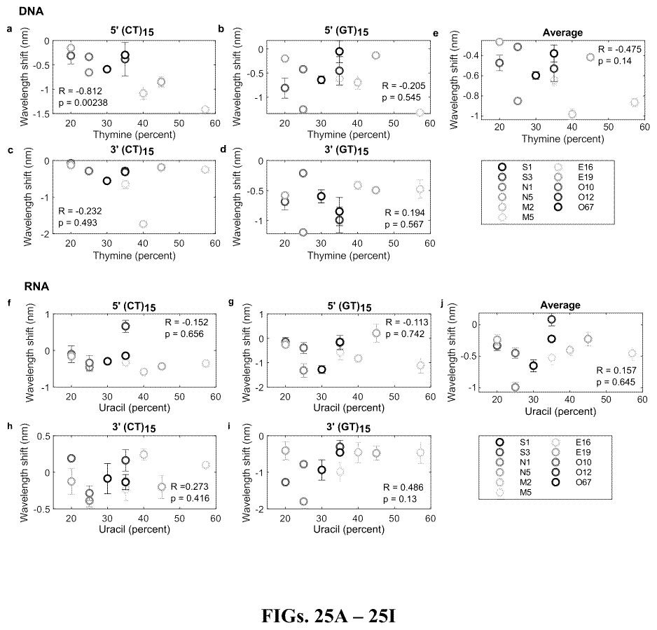

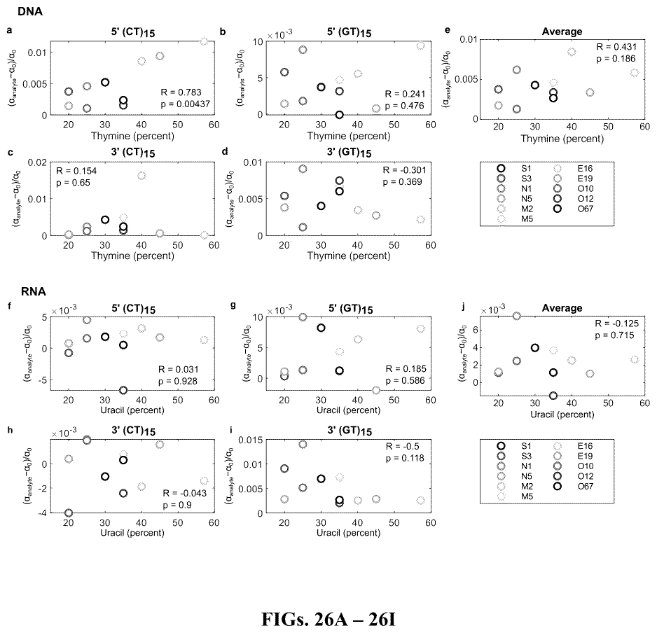

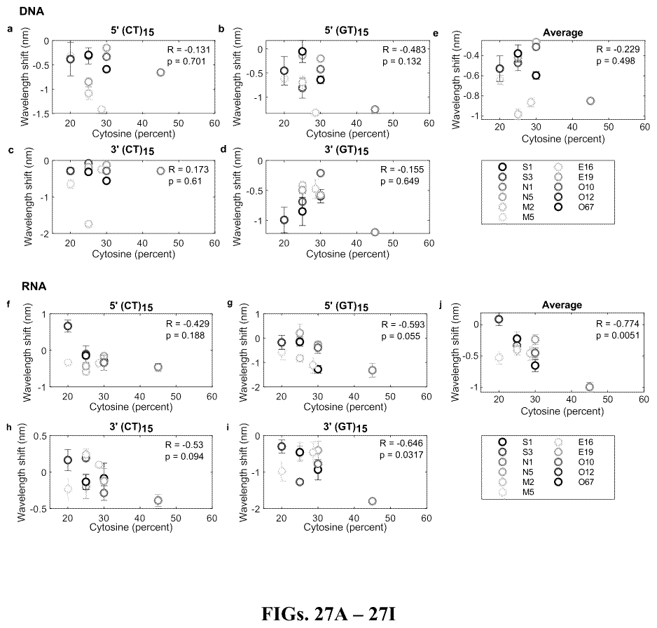

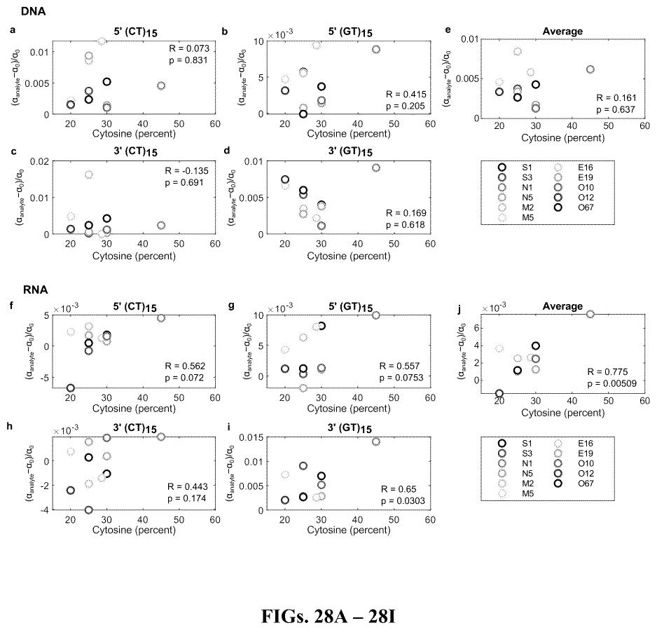

TABLE 6
Summary of Pearson's correlation coefficient (R) and p-value (P) of correlations
between analyte properties and SWCNT photophysical property changes

| Analyte | | CP | Solvatochromic | Normalized surface | |
|---|---|---|---|---|---|
| property | Analyte | anchor | shift | coverage change | FIG. |
| N_{Dm} | cDNA | 5′ (GT)₁₅ | R = 0.832 | R = −0.822 | FIG. 21B, |
| | | | P = 0.00148 | P = 0.0019 | FIG. 22B |
| N_{Dm} | cRNA | 5′ (GT)₁₅ | R = 0.653 | R = −0.678 | FIG. 21G, |
| | | | P = 0.0294 | P = 0.022 | FIG. 22G |
| ΔG_{Hp} | cDNA | 5′ (CT)₁₅ | R = −0.871 | R = 0.733 | FIG. 9A, |
| | | | P = 0.001 | P = 0.016 | FIG. 10A |
| L_{Min} | cRNA | 3′ (GT)₁₅ | R = −0.703 | R = 0.727 | FIG. 19I, |
| | | | P = 0.0158 | P = 0.0112 | FIG. 20I |
| Thymine | cDNA | 5′ (CT)₁₅ | R = −0.812 | R = 0.783 | FIG. 25A, |
| content | | | P = 0.00238 | P = 0.00437 | FIG. 26A |
| Cytosine | cRNA | 3′ (GT)₁₅ | R = −0.646 | R = 0.65 | FIG. 27I, |
| content | | | P = 0.0317 | P = 0.0303 | FIG. 28I |

From the correlations in Table 6, secondary structure within analyte strands negatively impacts its capability to hybridize with ssDNA-SWCNT. The negative correlation between N_{Dm} and normalized surface coverage change indicates that increased self-dimer formation disfavors SWCNT surface adsorption. Similarly, hairpin formation disfavors SWCNT surface adsorption as shown by the positive correlation between ΔG_{Hp} and relative change of DNA surface coverage. Additionally, L_{Min} correlations suggested that the loss of single-stranded regions to secondary structures adversely impacts SWCNT surface hybridization.

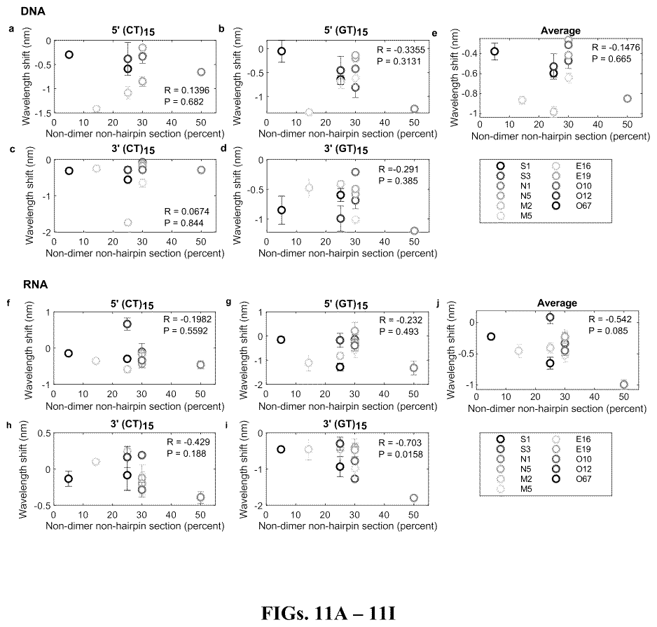

Next, we looked into the effects of individual nucleotide on PL responses. Both T and C content in the analyte sequences are positively correlated to PL responses. Previously, a single nucleotide SWCNT binding study showed a preference ordering of A>G>T>C.⁷⁵ Analyte T/C content translates to higher CP A/G content, which should more aggressively adsorb analyte strands to the SWCNT surface, leading to denser packing. Other analyte properties did not demonstrate statistically significant correlations (FIGS. 11-18, 23, 24, 29, 30).

### Condition Dependent Analyte Responses

The fact that both cDNA and cRNA generated selective responses demonstrated the versatility of our approach. The sequences that had the largest PL responses are M2-(CT)₁₅ and (CT)₁₅-E16 for cDNA and N1-(GT)₁₅ and S3-(GT)₁₅ for cRNA. We chose these constructs to study their potential as photophysical sensors. Considering that hybridization events occur on the ssDNA-SWCNT CP, we explored 2 methods that potentially can affect CP rearrangement during binding events: 1) bath sonication, and 2) surfactant.

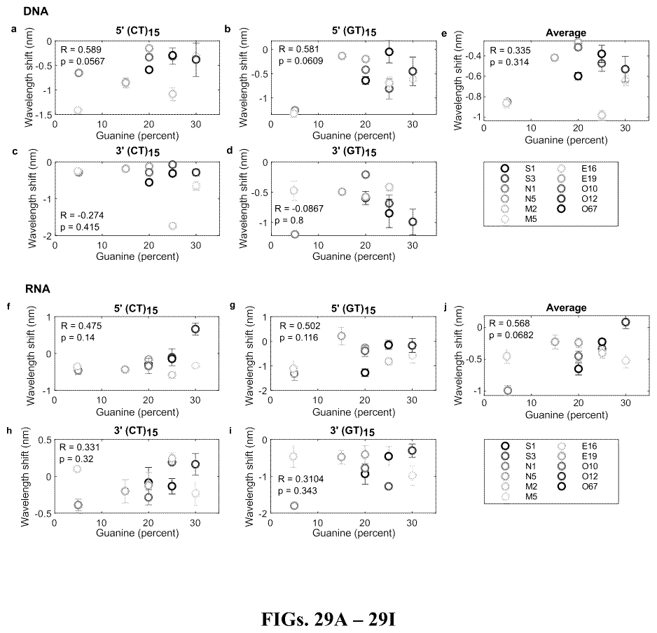

For the bath sonication treatment studies, we hypothesize that pre-analyte ultrasonication could prime the CP, and post-analyte ultrasonication can improve the slow kinetics of the CP rearrangement. Ultrasonicator power, water levels, sample volume and sample location within the bath were all kept constant to control for variability. M2-(CT)₁₅ was used to study ultrasonication effects. In general bath sonication up to 30 mins did not significantly alter PL responses in any configuration (FIGS. 4A-4C). In corresponding surface coverage results, post-incubation sonication of 20 or 30 mins showed increased surface coverage for complementary analytes. Unfortunately, surface coverage also increased for PBS and random controls. For the associated RNA experiments, we made similar observations (FIGS. 29A-29D). Overall, this bath sonication approach would confound results when used for sensing purposes.

For the surfactant treatment studies, we followed up on previous work showing sodium dodecylbenzene sulfonate (SDBS)⁷⁹ and sodium dodecyl sulfate (SDS)⁸⁰ addition improved PL responses. These amphiphilic molecules are commonly used to debundle SWCNTs by adsorbing to the SWCNT surface (benzene ring and long alkyl chain for SDBS and long alkyl chain for SDS) and interfacing with the aqueous solution via the small hydrophilic sulfonate head group. Addition of SDBS³¹ and SDS³² to ssDNA-SWCNT sensor dispersions previously enhanced the hybridization-induced wavelength modulation without losing sequence specificity. We used (CT)₁₅-E16-SWCNT to study these surfactant effects over a range of concentrations (0, 0.00125, 0.0025, 0.005, 0.01 and 0.02 w/v %) below the critical micelle concentrations. Two experimental conditions were tested, either with surfactant pre-incubation with ssDNA-SWCNT or co-incubation during the 1-hour analyte exposure.

SDBS co-incubation at low concentrations (0.0025%) showed a 94% increase in hypsochromic shift (FIG. 4E). However, increasing SDBS concentrations further resulted in PL response from non-complement analytes, eventually converging analyte responses to the same value. We attribute these results to SDBS replacing the adsorbed ssDNA on SWCNT surface and leaving the SWCNT CP more densely packed in a non-selective manner. The SDS addition experiments did not show any PL response improvements in the conditions tested (FIG. 4F, FIG. 4G).

The threshold surfactant concentrations where specific responses were retained were 0.0025% for SDBS and 0.005% for SDS, showing SDBS has a greater affinity for the SWCNT surface as expected.⁸¹ To summarize, in our experimental design, surfactant addition did not show significant improvement in transducing hybridization events, and were not employed further due to confounding effects.

### Detection Limit, Specificity and Compatibility in Biofluid

We tested the sensor response over a range of analyte concentrations and constructed a dose-response curve to determine the detection limit. Again, the best two DNA (M2-(CT)₁₅-SWCNT and (CT)₁₅-E16-SWCNT) and RNA (N1-(GT)₁₅-SWCNT and S3-(GT)₁₅-SWCNT) detecting constructs were chosen (FIG. 5A). A range of analyte concentrations from 0.1 nM to 1000 nM were chosen. Signal saturation occurred at 100-1000 nM (FIG. 5B). Non-complimentary random controls showed overall no significant response. The equilibrium constant K=k_{f}/k_{b} defined earlier were determined from the aforementioned Langmuir binding model for each construct.⁸²⁻⁸⁴ Rearranging Eqn. 5, the energy shift at each analyte concentration after incubation can be written as

$$Δ E = β \frac{{C}_{A} K}{1 + {C}_{A} K} \qquad ( 9 )$$

where β is a constant that summarizes all the constant terms in ΔEₘₐₓ(1−e^{-(k}^{_{f}}^{C}^{_{A}}^{+k}^{_{b}}^{)t}) and t is 1 hour for all analyte concentrations. The data in FIG. 5B were found to generate fits to Eqn. 9 with R²=0.983, 0.996, 0.955 and 0.96 for M2-(CT)₁₅, (CT)₁₅-E16, N1-(GT)₁₅ and S3-(GT)₁₅, respectively. The resulting kinetic parameters were α=2.692, 2.262, 1.921, and 0.833 meV, and the dissociation constant K_{D}=1/K=150.7, 181.3, 41, and 67.3 nM respectively (FIG. 5B). The limit of detection (LOD) is calculated using the formula LOD=S_{blank}+3σ_{blank}, where S_{blank} is the theoretical wavelength shift without the presence of analyte and σ_{blank} is the standard deviation of the response toward PBS buffer. The limits of detection assuming this binding model were 11, 56, 13 and 131 nM, respectively. In the context of viral RNA detection, the LODs were converted to be 12.8 log 10, 13.5 log 10, 12.9 log 10, 13.9 log 10 copies of viral genome with the assumption that each analyte has a single copy in the virus genome.

The specificity of the constructs toward non-complementary analytes was studied by comparing the responses of M2-(CT)₁₅, (CT)₁₅-E16, N1-(GT)₁₅ and S3-(GT)₁₅ toward the 10 other analyte sequences from Table 1 at 500 nM. Specificity appears to be retained (FIG. 5C).

Lastly, we assessed the compatibility of the ssDNA-SWCNT construct with complex biofluid as a connection to obvious medical applications. For the context of viral RNA detection from patient samples, saliva was chosen as the target media because an oral swab is not only easier to administer compared to blood or urine test but also more sensitive than the commonly employed nasal swab for diagnosis of asymptomatic and mild COVID-19 infection.⁸⁵ Saliva contains a variety of electrolytes, proteins, polypeptides, polynucleotides, and small organic substances that can perturb the SWCNT ssDNA CP and interfere with the solvatochromic response.⁸⁶ However, the electrolytes and carbohydrate-based matrix in saliva should have minimal adsorption onto typical nanoparticle surfaces.⁸⁷ Previous reports of SWCNT-phospholipid-based sensors for SARS-CoV-2 protein showed compatibility with saliva.⁸⁸ We introduced the commercially available saliva sample from pooled human donors at a final concentration of 1% v/v to the N1-(GT)₁₅ and S3-(GT)₁₅ construct dispersions. The PL shift amplitudes toward the target analytes showed no significant change between the PBS buffer condition and 1% v/v saliva. The response specificity over random control were also preserved (FIG. 5D), suggesting that the nanotube CP is agnostic to adsorption of saliva components at this saliva concentration to enable successful transduction of hybridization events.

### Description of Target Analyte Metrics Used in Correlations

We obtained all metrics from IDT's oligoanalyzer, which were calculated through well-established methods.⁹¹ These metrics are a way of decomposing analyte sequence into features for analysis. We studied the correlation of these features to our sensor responses to gain an understanding of which features influence the PL responses.
1) Most negative free energy of hairpin formation among all potential hairpins, ΔG_{Hp}. Hairpin formation in analyte create a loop structure that prevents it from hybridizing onto the SWCNT CP. The likelihood of formation of a specific hairpin is dictated by its free energy of formation. We use the most negative free energy of formation among all hairpins as a metric to gauge the likelihood of hairpin formation in the analyte sequence.
2) % length of the shortest single-stranded section among all hairpins, L_{Hp}. We calculate this metric as it indicates the availability of the analyte for hybridization even after hairpin formation. A long single-stranded section should motivate hybridization with ssDNA in SWCNT CP.
3) Free energy of hybridization, ΔG_{Hyb}. More negative free energy of hybridization indicates a greater driving force for the analyte to hybridize with its complement sequence.
4) Most negative free energy of self-dimerization among all potential self-dimers, ΔG_{Dm}. Self-dimer is the most prevalent form of secondar structures in oligonucleotide in bulk solution with highly negative free energy of formation. In the current experimental condition, the analyte is present at higher concentration than the adsorbed ssDNA in SWCNT CP, which further motivates self-dimerization over binding events on nanotube surface. We use the most negative free energy of formation among all self-dimers as a metric to gauge the likelihood of self-dimerization in the analyte sequence.
5) % length of the single-stranded section of the self-dimer in (4), L Dm. We select this metric for the same reason for metric (2).
6) Minimum of (2) and (5), L_{Min}. We select this metric for the same reason for metric (2) and (5).
7) Number of unique self-dimers possible, N_{Dm}. We use this metrics as an indication of the likelihood of self-dimer formation. Most self-dimers possess highly negative free energy of formation. The greater number of available dimer forms should indicate higher likelihood of self-dimerization.
8) A, T, G, C content individually. Analyte nucleotide composition may play a role in influencing adsorption to nanotube surface and thus hybridization to SWCNT CP.

### CONCLUSIONS

Functionalizing SWCNT with ssDNA represents a versatile and intuitive approach for the detection of single stranded DNA and RNA oligonucleotides through hybridization and modulation of the SWCNT fluorescence signal. In this work, we systematically studied nucleotide hybridization on SWCNT CP using SARS-COV-2 sequences as model analyte targets. Using a model of SWCNT solvatochromism, we explained the observed PL changes as a modulation in SWCNT surface ssDNA coverage following complementary analyte addition. We find that hybridization on the SWCNT surface has a lower enthalpy (−11.9 kJ mol⁻¹) than in the solution phase (−707 kJ mol⁻¹). We also validated a previous approach by attaching an anchor region to the recognition region, which significantly improved PL response and selectivity, with (GT)₁₅ anchors superior to others tested. By correlating analyte sequence features to PL responses, we found that secondary structures like hairpins and self-dimers are barriers to hybridization, suggesting that target sequence design is important for the overall detection process. We also varied incubation conditions to improve PL responses, bath sonication and surfactant additions. Both showed ineffective improvements. Finally, the best ssDNA CP candidates demonstrate biocompatibility in complex media. The results of this study significantly improve the understanding of nanotube ssDNA CP interactions with solution phase oligonucleotides.

The current study focuses on optimizing the sensor performance in vitro by investigating optimal sensor design and operating conditions. For application of the sensor to detect viral genomes in patient samples, future work should be directed at improving the detection limit. The LODs of the best DNA and RNA detection constructs, when used in the solution phase as demonstrated in this study, in this work need to be improved by 7 orders of magnitude to detect the SARS-CoV-2 viral genome in patient saliva samples (5.2 log 10 copies per mL).⁸⁹ When immobilized and probed at the single particle level, SWCNT sensors have been shown to resolve down to single molecule detection limits.⁷² Hence, a hardware design that allows massively parallel monitoring of single SWCNT fluorescent sensors should address this concern. As another strategy to improve the detection limit, an RNA amplification technique could be implemented prior to the sensor assay. Loop-mediated isothermal amplification (LAMP) is an excellent candidate due to its speed and simplicity. It requires minimal sample purification from the crude sample, takes place at isothermal conditions eliminating the need of expensive thermal cyclers and, most importantly, creates products with long single-stranded loops of up to 100-mer which can function as the target sites.⁹⁰ As the dsDNA experiments have shown that the ssDNA-SWCNT is only compatible with single strand oligonucleotide, the LAMP assay should be designed such that the single-stranded loops contain the target region for recognition. We demonstrate that this technique is agnostic to proteins and nucleotides present in biofluids and potentially remains so to LAMP assay enzymes. The versatility of this method and our findings pave the way for the rational design of ssDNA-SWCNT sensors against nucleotide targets, with potential implications for infectious disease management.

It should be understood that the subject matter defined in the appended claims is not necessarily limited to the specific implementations described above. The specific implementations described above are disclosed as examples only.

### CITATIONS

(1) Tenover, F. C. DNA Hybridization Techniques and Their Application to the Diagnosis of Infectious Diseases. Infect. Dis. Clin. North Am. 1993, 7 (2), 171-181.
(2) Sklar, J. DNA Hybridization in Diagnostic Pathology. Hum. Pathol. 1985, 16 (7), 654-658.
(3) Chen, L.; Zhang, J.; Lin, Z.; Zhang, Z.; Mao, M.; Wu, J.; Li, Q.; Zhang, Y.; Fan, C. Pharmaceutical Applications of Framework Nucleic Acids. Acta Pharm. Sin. B 2022, 12 (1), 76-91.
(4) Usher, P. A.; Galsgaard, E. D.; Kruse, K.; Wang, J.; Krogh, B. O.; Mandelbaum, J.; Almholt, K. Sensitive and Specific In Situ Hybridization for Early Drug Discovery BT—In Situ Hybridization Protocols; Nielsen, B. S., Ed.; Springer New York: New York, NY, 2014; pp 103-123.
(5) Rich, A. A Hybrid Helix Containing Both Deoxyribose and Ribose Polynucleotides and Its Relation to the Transfer of Information between the Nucleic Acids. Proc. Natl. Acad. Sci. 1960, 46 (8), 1044-1053.
(6) Drmanac, R.; Drmanac, S.; Strezoska, Z.; Paunesku, T.; Labat, I.; Zeremski, M.; Snoddy, J.; Funkhouser, W. K.; Koop, B.; et al. DNA Sequence Determination by Hybridization: A Strategy for Efficient Large-Scale Sequencing. Science (80-.). 1993, 260 (5114), 1649-1652.
(7) Vikrant, K.; Bhardwaj, N.; Bhardwaj, S. K.; Kim, K. H.; Deep, A. Nanomaterials as Efficient Platforms for Sensing DNA. Biomaterials 2019, 214, 119215.
(8) Jayaraman, A.; Hall, C. K.; Genzer, J. Computer Simulation Study of Probe-Target Hybridization in Model DNA Microarrays: Effect of Probe Surface Density and Target Concentration. J. Chem. Phys. 2007, 127 (14), 10B610.
(9) Tymoczko, J.; Schuhmann, W.; Gebala, M. Electrical Potential-Assisted DNA Hybridization. How to Mitigate Electrostatics for Surface DNA Hybridization. ACS Appl. Mater. Interfaces 2014, 6 (24), 21851-21858.
(10) Peterson, A. W.; Heaton, R. J.; Georgiadis, R. M. The Effect of Surface Probe Density on DNA Hybridization. Nucleic Acids Res. 2001, 29 (24), 5163-5168.
(11) Shchepinov, M. S.; Case-Green, S. C.; Southern, E. M. Steric Factors Influencing Hybridisation of Nucleic Acids to Oligonucleotide Arrays. Nucleic Acids Res. 1997, 25 (6), 1155-1161.
(12) Watterson, J. H.; Piunno, P. A. E.; Wust, C. C.; Krull, U. J. Effects of Oligonucleotide Immobilization Density on Selectivity of Quantitative Transduction of Hybridization of Immobilized DNA. Langmuir 2000, 16 (11), 4984-4992.
(13) Vainrub, A.; Montgomery Pettitt, B. Surface Electrostatic Effects in Oligonucleotide Microarrays: Control and Optimization of Binding Thermodynamics. Biopolym. Orig. Res. Biomol. 2003, 68 (2), 265-270.
(14) Traeger, J. C.; Schwartz, D. K. Surface-Mediated DNA Hybridization: Effects of DNA Conformation, Surface Chemistry, and Electrostatics. Langmuir 2017, 33 (44), 12651-12659.
(15) Wong, I. Y.; Melosh, N. A. An Electrostatic Model for DNA Surface Hybridization. Biophys. J. 2010, 98 (12), 2954-2963.
(16) Glazer, M. I.; Fidanza, J. A.; McGall, G. H.; Trulson, M. O.; Forman, J. E.; Frank, C. W. Kinetics of Oligonucleotide Hybridization to DNA Probe Arrays on High-Capacity Porous Silica Substrates. Biophys. J. 2007, 93 (5), 1661-1676.
(17) Tawa, K.; Knoll, W. Mismatching Base-pair Dependence of the Kinetics of DNA-DNA Hybridization Studied by Surface Plasmon Fluorescence Spectroscopy. Nucleic Acids Res. 2004, 32 (8), 2372-2377.
(18) Hooyberghs, J.; Van Hummelen, P.; Carlon, E. The Effects of Mismatches on Hybridization in DNA Microarrays: Determination of Nearest Neighbor Parameters. Nucleic Acids Res. 2009, 37 (7), e53-e53.
(19) Letowski, J.; Brousseau, R.; Masson, L. Designing Better Probes: Effect of Probe Size, Mismatch Position and Number on Hybridization in DNA Oligonucleotide Microarrays. J. Microbiol. Methods 2004, 57 (2), 269-278.
(20) Stillman, B. A.; Tonkinson, J. L. Expression Microarray Hybridization Kinetics Depend on Length of the Immobilized DNA but Are Independent of Immobilization Substrate. Anal. Biochem. 2001, 295 (2), 149-157.
(21) Halperin, A.; Buhot, A.; Zhulina, E. B. Brush Effects on DNA Chips: Thermodynamics, Kinetics, and Design Guidelines. Biophys. J. 2005, 89 (2), 796-811.
(22) Sedighi, A.; Li, P. C. H.; Pekcevik, I. C.; Gates, B. D. A Proposed Mechanism of the Influence of Gold Nanoparticles on DNA Hybridization. ACS Nano 2014, 8 (7), 6765-6777.
(23) Schneider, T.; Jahr, N.; Jatschka, J.; Csaki, A.; Stranik, O.; Fritzsche, W. Localized Surface Plasmon Resonance (LSPR) Study of DNA Hybridization at Single Nanoparticle Transducers. J. Nanoparticle Res. 2013, 15 (4), 1531.
(24) Dugas, V.; Depret, G.; Chevalier, Y.; Nesme, X.; Souteyrand, É. Immobilization of Single-Stranded DNA Fragments to Solid Surfaces and Their Repeatable Specific Hybridization: Covalent Binding or Adsorption? Sensors Actuators B Chem. 2004, 101 (1), 112-121.
(25) Wang, J.; Wang, L.; Liu, X.; Liang, Z.; Song, S.; Li, W.; Li, G.; Fan, C. A Gold Nanoparticle-based Aptamer Target Binding Readout for ATP Assay. Adv. Mater. 2007, 19 (22), 3943-3946.
(26) Liu, B.; Liu, J. Interface-Driven Hybrid Materials Based on DNA-Functionalized Gold Nanoparticles. Matter 2019, 1 (4), 825-847.
(27) Elghanian, R.; Storhoff, J. J.; Mucic, R. C.; Letsinger, R. L.; Mirkin, C. A. Selective Colorimetric Detection of Polynucleotides Based on the Distance-Dependent Optical Properties of Gold Nanoparticles. Science (80-.). 1997, 277 (5329), 1078-1081.
(28) Li, H.; Rothberg, L. Colorimetric Detection of DNA Sequences Based on Electrostatic Interactions with Unmodified Gold Nanoparticles. Proc. Natl. Acad. Sci. 2004, 101 (39), 14036-14039.
(29) Sorgenfrei, S.; Chiu, C.; Gonzalez, R. L.; Yu, Y. J.; Kim, P.; Nuckolls, C.; Shepard, K. L. Label-Free Single-Molecule Detection of DNA-Hybridization Kinetics with a Carbon Nanotube Field-Effect Transistor. Nat. Nanotechnol. 2011, 6 (2), 126-132.
(30) Barnes, B.; Wang, P.; Wang, Y. Parallel Field-Effect Nanosensors Detect Trace Biomarkers Rapidly at Physiological High-Ionic-Strength Conditions. ACS Sensors 2022, 7 (9), 2537-2544.
(31) Harvey, J. D.; Jena, P. V; Baker, H. A.; Zerze, G. H.; Williams, R. M.; Galassi, T. V; Roxbury, D.; Mittal, J.; Heller, D. A. A Carbon Nanotube Reporter of MicroRNA Hybridization Events in Vivo. Nat. Biomed. Eng. 2017, 1 (4), 41.
(32) Harvey, J. D.; Baker, H. A.; Ortiz, M. V; Kentsis, A.; Heller, D. A. HIV Detection via a Carbon Nanotube RNA Sensor. ACS Sensors 2019, 4 (5), 1236-1244.
(33) Loo, A. H.; Sofer, Z.; Bousa, D.; Ulbrich, P.; Bonanni, A.; Pumera, M. Carboxylic Carbon Quantum Dots as a Fluorescent Sensing Platform for DNA Detection. ACS Appl. Mater. Interfaces 2016, 8 (3), 1951-1957.
(34) Qian, Z. S.; Shan, X. Y.; Chai, L. J.; Ma, J. J.; Chen, J. R.; Feng, H. A Universal Fluorescence Sensing Strategy Based on Biocompatible Graphene Quantum Dots and Graphene Oxide for the Detection of DNA. Nanoscale 2014, 6 (11), 5671-5674.
(35) Ye, Y. D.; Xia, L.; Xu, D. D.; Xing, X. J.; Pang, D. W.; Tang, H. W. DNA-Stabilized Silver Nanoclusters and Carbon Nanoparticles Oxide: A Sensitive Platform for Label-Free Fluorescence Turn-on Detection of HIV-DNA Sequences. Biosens. Bioelectron. 2016, 85, 837-843.
(36) Godavarthi, S.; Kumar, K. M.; Vélez, E. V.; Hernandez-Eligio, A.; Mahendhiran, M.; Hernandez-Como, N.; Aleman, M.; Gomez, L. M. Nitrogen Doped Carbon Dots Derived from Sargassum Fluitans as Fluorophore for DNA Detection. J. Photochem. Photobiol. B Biol. 2017, 172, 36-41.
(37) Qiu, W.; Xu, H.; Takalkar, S.; Gurung, A. S.; Liu, B.; Zheng, Y.; Guo, Z.; Baloda, M.; Baryeh, K.; et al. Carbon Nanotube-Based Lateral Flow Biosensor for Sensitive and Rapid Detection of DNA Sequence. Biosens. Bioelectron. 2015, 64, 367-372.
(38) Lee, M. A.; Wang, S.; Jin, X.; Bakh, N. A.; Nguyen, F. T.; Dong, J.; Silmore, K. S.; Gong, X.; Pham, C.; et al. Implantable Nanosensors for Human Steroid Hormone Sensing In Vivo Using a Self-Templating Corona Phase Molecular Recognition. Adv. Healthc. Mater. 2020, 9 (21), 2000429.
(39) Kruss, S.; Hilmer, A. J.; Zhang, J.; Reuel, N. F.; Mu, B.; Strano, M. S. Carbon Nanotubes as Optical Biomedical Sensors. Adv. Drug Deliv. Rev. 2013, 65 (15), 1933-1950.
(40) O'connell, M. J.; Bachilo, S. M.; Huffman, C. B.; Moore, V. C.; Strano, M. S.; Haroz, E. H.; Rialon, K. L.; Boul, P. J.; Noon, W. H.; et al. Band Gap Fluorescence from Individual Single-Walled Carbon Nanotubes. Science (80-.). 2002, 297 (5581), 593-596.
(41) Bachilo, S. M.; Strano, M. S.; Kittrell, C.; Hauge, R. H.; Smalley, R. E.; Weisman, R. B. Structure-Assigned Optical Spectra of Single-Walled Carbon Nanotubes. Science (80-.). 2002, 298 (5602), 2361-2366.
(42) Wray, S.; Cope, M.; Delpy, D. T.; Wyatt, J. S.; Reynolds, E. O. R. Characterization of the near Infrared Absorption Spectra of Cytochrome Aa3 and Haemoglobin for the Non-Invasive Monitoring of Cerebral Oxygenation. Biochim. Biophys. Acta—Bioenerg. 1988, 933 (1), 184-192.
(43) Cheong, W. F.; Prahl, S. A.; Welch, A. J. A Review of the Optical Properties of Biological Tissues. IEEE J. Quantum Electron. 1990, 26 (12), 2166-2185.
(44) Weissleder, R.; Ntziachristos, V. Shedding Light onto Live Molecular Targets. Nat. Med. 2003, 9 (1), 123-128.
(45) Wang, F.; Dukovic, G.; Brus, L. E.; Heinz, T. F. The Optical Resonances in Carbon Nanotubes Arise from Excitons. Science (80-.). 2005, 308 (5723), 838-841.
(46) Heller, D. A.; Baik, S.; Eurell, T. E.; Strano, M. S. Single-Walled Carbon Nanotube Spectroscopy in Live Cells: Towards Long-Term Labels and Optical Sensors. Adv. Mater. 2005, 17 (23), 2793-2799.
(47) Lew, T. T. S.; Park, M.; Cui, J.; Strano, M. S. Plant Nanobionic Sensors for Arsenic Detection. Adv. Mater. 2021, 33 (1), 2005683.
(48) Zhang, J.; Landry, M. P.; Barone, P. W.; Kim, J. H.; Lin, S.; Ulissi, Z. W.; Lin, D.; Mu, B.; Boghossian, A. A.; et al. Molecular Recognition Using Corona Phase Complexes Made of Synthetic Polymers Adsorbed on Carbon Nanotubes. Nat. Nanotechnol. 2013, 8 (12), 959-968.
(49) Cognet, L.; Tsyboulski, D. A.; Rocha, J. D. R.; Doyle, C. D.; Tour, J. M.; Weisman, R. B. Stepwise Quenching of Exciton Fluorescence in Carbon Nanotubes by Single-Molecule Reactions. Science (80-.). 2007, 316 (5830), 1465-1468.
(50) Jin, H.; Heller, D. A.; Kalbacova, M.; Kim, J. H.; Zhang, J.; Boghossian, A. A.; Maheshri, N.; Strano, M. S. Detection of Single-Molecule H2O2 Signalling from Epidermal Growth Factor Receptor Using Fluorescent Single-Walled Carbon Nanotubes. Nat. Nanotechnol. 2010, 5 (4), 302-309.
(51) Choi, J. H.; Strano, M. S. Solvatochromism in Single-Walled Carbon Nanotubes. Appl. Phys. Lett. 2007, 90 (22), 223114.
(52) Kruss, S.; Landry, M. P.; Vander Ende, E.; Lima, B. M. A.; Reuel, N. F.; Zhang, J.; Nelson, J.; Mu, B.; Hilmer, A.; et al. Neurotransmitter Detection Using Corona Phase Molecular Recognition on Fluorescent Single-Walled Carbon Nanotube Sensors. J. Am. Chem. Soc. 2014, 136 (2), 713-724.
(53) Jeng, E. S.; Moll, A. E.; Roy, A. C.; Gastala, J. B.; Strano, M. S. Detection of DNA Hybridization Using the Near-Infrared Band-Gap Fluorescence of Single-Walled Carbon Nanotubes. Nano Lett. 2006, 6 (3), 371-375.
(54) Jeong, S.; Gonzilez-Grandio, E.; Navarro, N.; Pinals, R. L.; Ledesma, F.; Yang, D.; Landry, M. P. Extraction of Viral Nucleic Acids with Carbon Nanotubes Increases SARS-CoV-2 Quantitative Reverse Transcription Polymerase Chain Reaction Detection Sensitivity. ACS Nano 2021, 15 (6), 10309-10317.
(55) Wu, S. J.; Schuergers, N.; Lin, K. H.; Gillen, A. J.; Corminboeuf, C.; Boghossian, A. A. Restriction Enzyme Analysis of Double-Stranded DNA on Pristine Single-Walled Carbon Nanotubes. ACS Appl. Mater. Interfaces 2018, 10 (43), 37386-37395.
(56) Toyofuku, R.; Ohura, S.; Ito, M.; Homma, Y.; Umemura, K. Interactions of Secondary DNA and Initial DNA on Single-Walled Carbon Nanotube Surfaces Studied by Photoluminescence, Atomic Force Microscopy, and Electrophoresis. J. Nanomater. 2019, 2019, 2875439.
(57) Markham, N. R.; Zuker, M. UNAFold BT—Bioinformatics: Structure, Function and Applications; Keith, J. M., Ed.; Humana Press: Totowa, NJ, 2008; pp 3-31.
(58) Nakashima, N.; Okuzono, S.; Murakami, H.; Nakai, T.; Yoshikawa, K. DNA Dissolves Single-Walled Carbon Nanotubes in Water. Chem. Lett. 2003, 32 (5), 456-457.
(59) Zheng, M.; Jagota, A.; Semke, E. D.; Diner, B. A.; Mclean, R. S.; Lustig, S. R.; Richardson, R. E.; Tassi, N. G. DNA-Assisted Dispersion and Separation of Carbon Nanotubes. Nat. Mater. 2003, 2 (5), 338-342.
(60) Salem, D. P.; Gong, X.; Liu, A. T.; Koman, V. B.; Dong, J.; Strano, M. S. Ionic Strength-Mediated Phase Transitions of Surface-Adsorbed DNA on Single-Walled Carbon Nanotubes. J. Am. Chem. Soc. 2017, 139 (46), 16791-16802.
(61) Zhang, J.; Kruss, S.; Hilmer, A. J.; Shimizu, S.; Schmois, Z.; De La Cruz, F.; Barone, P. W.; Reuel, N. F.; Heller, D. A.; et al. A Rapid, Direct, Quantitative, and Label-Free Detector of Cardiac Biomarker Troponin T Using Near-Infrared Fluorescent Single-Walled Carbon Nanotube Sensors. Adv. Healthc. Mater. 2014, 3 (3), 412-423.
(62) Gan, H. H.; Pasquali, S.; Schlick, T. Exploring the Repertoire of RNA Secondary Motifs Using Graph Theory; Implications for RNA Design. Nucleic Acids Res. 2003, 31 (11), 2926-2943.
(63) Wan, Y.; Kertesz, M.; Spitale, R. C.; Segal, E.; Chang, H. Y. Understanding the Transcriptome through RNA Structure. Nat. Rev. Genet. 2011, 12 (9), 641-655.
(64) Gong, X.; Renegar, N.; Levi, R.; Strano, M. S. Machine Learning for the Discovery of Molecular Recognition Based on Single-Walled Carbon Nanotube Corona-Phases. npj Comput. Mater. 2022, 8 (1), 135.
(65) Chen, C.; Wang, W.; Wang, Z.; Wei, F.; Zhao, X. S. Influence of Secondary Structure on Kinetics and Reaction Mechanism of DNA Hybridization. Nucleic Acids Res. 2007, 35 (9), 2875-2884.
(66) Owczarzy, R.; Tataurov, A. V; Wu, Y.; Manthey, J. A.; McQuisten, K. A.; Almabrazi, H. G.; Pedersen, K. F.; Lin, Y.; Garretson, J.; et al. IDT SciTools: A Suite for Analysis and Design of Nucleic Acid Oligomers. Nucleic Acids Res. 2008, 36 (Web Server issue), W163-W169.
(67) Gong, X.; Park, M.; Parviz, D.; Silmore, K. S.; Gordiichuk, P.; Lew, T. T. S.; Strano, M. S. Single-Particle Tracking for Understanding Polydisperse Nanoparticle Dispersions. Small 2019, 15 (37), 1901468.
(68) Marky, L. A.; Breslauer, K. J. Calculating Thermodynamic Data for Transitions of Any Molecularity from Equilibrium Melting Curves. Biopolym. Orig. Res. Biomol. 1987, 26 (9), 1601-1620.
(69) Wu, P.; Sugimoto, N. Transition Characteristics and Thermodynamic Analysis of DNA Duplex Formation: A Quantitative Consideration for the Extent of Duplex Association. Nucleic Acids Res. 2000, 28 (23), 4762-4768.
(70) Dimitrov, R. A.; Zuker, M. Prediction of Hybridization and Melting for Double-Stranded Nucleic Acids. Biophys. J. 2004, 87 (1), 215-226.
(71) John, S. A Unified View of Polymer, Dumbbell, and Oligonucleotide DNA Nearest-Neighbor Thermodynamics. Proc. Natl. Acad. Sci. 1998, 95 (4), 1460-1465.
(72) Bisker, G.; Dong, J.; Park, H. D.; Iverson, N. M.; Ahn, J.; Nelson, J. T.; Landry, M. P.; Kruss, S.; Strano, M. S. Protein-Targeted Corona Phase Molecular Recognition. Nat. Commun. 2016, 7 (1), 10241.
(73) Heller, D. A.; Jeng, E. S.; Yeung, T. K.; Martinez, B. M.; Moll, A. E.; Gastala, J. B.; Strano, M. S. Optical Detection of DNA Conformational Polymorphism on Single-Walled Carbon Nanotubes. Science (80-.). 2006, 311 (5760), 508-511.
(74) Park, M.; Salem, D. P.; Parviz, D.; Gong, X.; Silmore, K. S.; Lew, T. T. S.; Khong, D. T.; Ang, M. C. Y.; Kwak, S. Y.; et al. Measuring the Accessible Surface Area within the Nanoparticle Corona Using Molecular Probe Adsorption. Nano Lett. 2019, 19 (11), 7712-7724.
(75) Iliafar, S.; Mittal, J.; Vezenov, D.; Jagota, A. Interaction of Single-Stranded DNA with Curved Carbon Nanotube Is Much Stronger Than with Flat Graphite. J. Am. Chem. Soc. 2014,136 (37), 12947-12957.
(76) Das, A.; Sood, A. K.; Maiti, P. K.; Das, M.; Varadarajan, R.; Rao, C. N. R. Binding of Nucleobases with Single-Walled Carbon Nanotubes: Theory and Experiment. Chem. Phys. Lett. 2008, 453 (4), 266-273.
(77) Akdim, B.; Pachter, R.; Day, P. N.; Kim, S. S.; Naik, R. R. On Modeling Biomolecular-Surface Nonbonded Interactions: Application to Nucleobase Adsorption on Single-Wall Carbon Nanotube Surfaces. Nanotechnology 2012, 23 (16), 165703.
(78) Wang, Y. Theoretical Evidence for the Stronger Ability of Thymine to Disperse SWCNT than Cytosine and Adenine: Self-Stacking of DNA Bases vs Their Cross-Stacking with SWCNT. J. Phys. Chem. C 2008, 112 (37), 14297-14305.
(79) Priya, B. R.; Byrne, H. J. Investigation of Sodium Dodecyl Benzene Sulfonate Assisted Dispersion and Debundling of Single-Wall Carbon Nanotubes. J. Phys. Chem. C 2008, 112 (2), 332-337.
(80) Richard, C.; Balavoine, F.; Schultz, P.; Ebbesen, T. W.; Mioskowski, C. Supramolecular Self-Assembly of Lipid Derivatives on Carbon Nanotubes. Science (80-.). 2003, 300 (5620), 775 LP-778.
(81) Islam, M. F.; Rojas, E.; Bergey, D. M.; Johnson, A. T.; Yodh, A. G. High Weight Fraction Surfactant Solubilization of Single-Wall Carbon Nanotubes in Water. Nano Lett. 2003, 3 (2), 269-273.
(82) Wetmur, J. G.; Davidson, N. Kinetics of Renaturation of DNA. J. Mol. Biol. 1968, 31 (3), 349-370.
(83) Bishop, J.; Blair, S.; Chagovetz, A. M. A Competitive Kinetic Model of Nucleic Acid Surface Hybridization in the Presence of Point Mutants. Biophys. J. 2006, 90 (3), 831-840.
(84) Halperin, A.; Buhot, A.; Zhulina, E. B. On the Hybridization Isotherms of DNA Microarrays: The Langmuir Model and Its Extensions. J. Phys. Condens. Matter 2006, 18 (18), S463.
(85) Teo, A. K. J.; Choudhury, Y.; Tan, I. B.; Cher, C. Y.; Chew, S. H.; Wan, Z. Y.; Cheng, L. T. E.; Oon, L. L. E.; Tan, M. H.; et al. Saliva Is More Sensitive than Nasopharyngeal or Nasal Swabs for Diagnosis of Asymptomatic and Mild COVID-19 Infection. Sci. Rep. 2021, 11 (1), 3134.
(86) Kubala, E.; Strzelecka, P.; Grzegocka, M.; Lietz-Kijak, D.; Gronwald, H.; Skomro, P.; Kijak, E. A Review of Selected Studies That Determine the Physical and Chemical Properties of Saliva in the Field of Dental Treatment. Biomed Res. Int. 2018, 2018, 6572381.
(87) Ngamchuea, K.; Batchelor-McAuley, C.; Compton, R. G. The Fate of Silver Nanoparticles in Authentic Human Saliva. Nanotoxicology 2018, 12 (4), 305-311.
(88) Cho, S. Y.; Jin, X.; Gong, X.; Yang, S.; Cui, J.; Strano, M. S. Antibody-Free Rapid Detection of SARS-CoV-2 Proteins Using Corona Phase Molecular Recognition to Accelerate Development Time. Anal. Chem. 2021, 93 (44), 14685-14693.
(89) To, K. K. W.; Tsang, O. T. Y.; Leung, W. S.; Tam, A. R.; Wu, T. C.; Lung, D. C.; Yip, C. C. Y.; Cai, J. P.; Chan, J. M. C.; et al. Temporal Profiles of Viral Load in Posterior Oropharyngeal Saliva Samples and Serum Antibody Responses during Infection by SARS-CoV-2: An Observational Cohort Study. Lancet Infect. Dis. 2020, 20 (5), 565-574.
(90) Sahoo, P. R.; Sethy, K.; Mohapatra, S.; Panda, D. Loop Mediated Isothermal Amplification: An Innovative Gene Amplification Technique for Animal Diseases. Vet. world 2016, 9 (5), 465-469.
(91) Owczarzy, R.; Tataurov, A. V; Wu, Y.; Manthey, J. A.; McQuisten, K. A.; Almabrazi, H. G.; Pedersen, K. F.; Lin, Y.; Garretson, J.; et al. IDT SciTools: A Suite for Analysis and Design of Nucleic Acid Oligomers. Nucleic Acids Res. 2008, 36 (Web Server issue), W163-W169.

1. A system for detecting an analyte in a sample comprising semiconducting single-walled carbon nanotubes (SWCNTs) and surface-adsorbed nucleic acids, wherein the surface-adsorbed nucleic acids comprise a complementary region and at least one anchor,
wherein the complementary region comprises a nucleic acid sequence that is complementary to and/or hybridizes to a target region of the analyte; and
wherein the at least one anchor comprises a nucleic acid sequence that is not complementary to the target region of the analyte.

2. The system of claim 1 wherein the surface-adsorbed nucleic acids are surface-adsorbed single-strand nucleic acids.

3. The system of claim 1 wherein the analyte comprises a nucleic acid selected from the group consisting of single strand DNA (ssDNA), micro RNA, and viral RNA.

4. The system of claim 1 wherein the surface-adsorbed nucleic acids are ssDNA.

5. The system of claim 1 comprising one anchor.

6. The system of claim 1 wherein the anchor is 5′ to the complementary region.

7. The system of claim 1 wherein the anchor is 3′ to the complementary region.

8. The system of claim 1 comprising two anchors, wherein one anchor is 5′ to the complementary region and one anchor is 3′ to the complementary region.

9. The system of claim 1, wherein each anchor comprises 6-80 nucleotides.

10. (canceled)

11. The system of claim 1 wherein the anchor comprises (GT)ₓ, or (CT)ₓ, where in x is 3-40.

12. (canceled)

13. The system of claim 1 wherein the dissociation constant of the complementary region for the target region of the analyte is about 5-20 nM.

14. (canceled)

15. The system of claim 1 wherein the analyte is a microbe or a virus.

16. (canceled)

17. The system of claim 15 wherein the analyte is a virus, and the virus is a SARS-CoV-2 virus, wherein the target region is selected from the region encoding the spike protein, the region encoding the membrane protein, the region encoding the nucleocapsid protein, the region encoding the envelope protein, a region encoding a non-functional protein, or a non-coding region.

18. (canceled)

19. The system of claim 1 wherein the surface-adsorbed nucleic acids are complementary to a sequence selected from a SARS-CoV-2 virus, wherein the sequence is selected from the region encoding the spike protein, the region encoding the membrane protein, the region encoding the nucleocapsid protein, the region encoding the envelope protein, a region encoding a non-functional protein, or a non-coding region.

20. The system of claim 1 wherein the sample is a biological sample selected from the group consisting of saliva, blood, urine, tissue, cells, and nasopharyngeal swabs.

21.-23. (canceled)

24. A method of detecting an analyte in a sample, the method comprising:
(i) providing a system according to claim 1 in solution;
(ii) combining the sample with the system in solution;
(iii) incubating the system and the sample; and
(iv) measuring the photoluminescence (PL) of the system;
wherein a shift in wavelength and/or intensity of the system relative to the system absent exposure to analyte indicates the presence of the analyte.

25.-26. (canceled)

27. The method of claim 24, wherein the PL is measured in the 850-1250 nm range.

28. The method of claim 24 wherein the analyte is a microbe or a virus.

29. (canceled)

30. The method of claim 28 wherein the analyte is a virus, and the virus is a SARS-CoV-2 virus, wherein the target region is selected from the region encoding the spike protein, the region encoding the membrane protein, the region encoding the nucleocapsid protein, the region encoding the envelope protein, a region encoding a non-functional protein, or a non-coding region.

31. (canceled)

32. The method of claim 24 wherein the surface-adsorbed nucleic acids are complementary to a sequence selected from a SARS-CoV-2 virus, wherein the sequence is selected from the region encoding the spike protein, the region encoding the membrane protein, the region encoding the nucleocapsid protein, the region encoding the envelope protein, a region encoding a non-functional protein, or a non-coding region.

33.-36. (canceled)


SEQUENCE LISTING (85 sequences)

SEQ ID NO: 1 - DNA, 20 bp, Severe Acute Respiratory Syndrome Coronavirus 2
  source 1..20: mol_type = genomic DNA
acactactgatgctgtccgt

SEQ ID NO: 2 - DNA, 20 bp, Severe Acute Respiratory Syndrome Coronavirus 2
  source 1..20: mol_type = genomic DNA
cctcaatgaggttgccaaga

SEQ ID NO: 3 - DNA, 20 bp, Severe Acute Respiratory Syndrome Coronavirus 2
  source 1..20: mol_type = genomic DNA
tcaccgctctcactcaacat

SEQ ID NO: 4 - DNA, 20 bp, Severe Acute Respiratory Syndrome Coronavirus 2
  source 1..20: mol_type = genomic DNA
cacgtagtcgcaacagttca

SEQ ID NO: 5 - DNA, 20 bp, Severe Acute Respiratory Syndrome Coronavirus 2
  source 1..20: mol_type = genomic DNA
ctttcagactgtttgcgcgt

SEQ ID NO: 6 - DNA, 20 bp, Severe Acute Respiratory Syndrome Coronavirus 2
  source 1..20: mol_type = genomic DNA
gtgactcaggttttgctgca

SEQ ID NO: 7 - DNA, 21 bp, Severe Acute Respiratory Syndrome Coronavirus 2
  source 1..21: mol_type = genomic DNA
ccttctttttacgtttactct

SEQ ID NO: 8 - DNA, 20 bp, Severe Acute Respiratory Syndrome Coronavirus 2
  source 1..20: mol_type = genomic DNA
ttcttctagagttcctgatc

SEQ ID NO: 9 - DNA, 20 bp, Severe Acute Respiratory Syndrome Coronavirus 2
  source 1..20: mol_type = genomic DNA
agtgtgcctattgggttcca

SEQ ID NO: 10 - DNA, 20 bp, Severe Acute Respiratory Syndrome Coronavirus 2
  source 1..20: mol_type = genomic DNA
tcaaccgctgctttaggtgt

SEQ ID NO: 11 - DNA, 20 bp, Severe Acute Respiratory Syndrome Coronavirus 2
  source 1..20: mol_type = genomic DNA
tgttcgcattcaaccaggac

SEQ ID NO: 12 - DNA, 22 bp, synthetic construct
  source 1..22: mol_type = other DNA
ggggaacttctcctgctagaat

SEQ ID NO: 13 - DNA, 52 bp, synthetic construct
  source 1..52: mol_type = other DNA
ctctctctctctctctctctctctctctctattctagcaggagaagttcccc

SEQ ID NO: 14 - DNA, 23 bp, synthetic construct
  source 1..23: mol_type = other DNA
gttacatgttcgttgggctcttc

SEQ ID NO: 15 - RNA, 20 nt, synthetic construct
  source 1..20: mol_type = other RNA
acactactgatgctgtccgt

SEQ ID NO: 16 - RNA, 20 nt, synthetic construct
  source 1..20: mol_type = other RNA
cctcaatgaggttgccaaga

SEQ ID NO: 17 - RNA, 20 nt, synthetic construct
  source 1..20: mol_type = other RNA
cacgtagtcgcaacagttca

SEQ ID NO: 18 - RNA, 20 nt, synthetic construct
  source 1..20: mol_type = other RNA
ctttcagactgtttgcgcgt

SEQ ID NO: 19 - RNA, 20 nt, synthetic construct
  source 1..20: mol_type = other RNA
gtgactcaggttttgctgca

SEQ ID NO: 20 - RNA, 21 nt, synthetic construct
  source 1..21: mol_type = other RNA
ccttctttttacgtttactct

SEQ ID NO: 21 - RNA, 20 nt, synthetic construct
  source 1..20: mol_type = other RNA
ttcttctagagttcctgatc

SEQ ID NO: 22 - RNA, 20 nt, synthetic construct
  source 1..20: mol_type = other RNA
agtgtgcctattgggttcca

SEQ ID NO: 23 - RNA, 20 nt, synthetic construct
  source 1..20: mol_type = other RNA
tcaaccgctgctttaggtgt

SEQ ID NO: 24 - RNA, 20 nt, synthetic construct
  source 1..20: mol_type = other RNA
tgttcgcattcaaccaggac

SEQ ID NO: 25 - DNA, 20 bp, synthetic construct
  source 1..20: mol_type = other DNA
acggacagcatcagtagtgt

SEQ ID NO: 26 - DNA, 20 bp, synthetic construct
  source 1..20: mol_type = other DNA
tcttggcaacctcattgagg

SEQ ID NO: 27 - DNA, 20 bp, synthetic construct
  source 1..20: mol_type = other DNA
atgttgagtgagagcggtga

SEQ ID NO: 28 - DNA, 20 bp, synthetic construct
  source 1..20: mol_type = other DNA
tgaactgttgcgactacgtg

SEQ ID NO: 29 - DNA, 20 bp, synthetic construct
  source 1..20: mol_type = other DNA
acgcgcaaacagtctgaaag

SEQ ID NO: 30 - DNA, 20 bp, synthetic construct
  source 1..20: mol_type = other DNA
tgcagcaaaacctgagtcac

SEQ ID NO: 31 - DNA, 21 bp, synthetic construct
  source 1..21: mol_type = other DNA
agagtaaacgtaaaaagaagg

SEQ ID NO: 32 - DNA, 20 bp, synthetic construct
  source 1..20: mol_type = other DNA
gatcaggaactctagaagaa

SEQ ID NO: 33 - DNA, 20 bp, synthetic construct
  source 1..20: mol_type = other DNA
gtcctggttgaatgcgaaca

SEQ ID NO: 34 - DNA, 20 bp, synthetic construct
  source 1..20: mol_type = other DNA
tggaacccaataggcacact

SEQ ID NO: 35 - DNA, 20 bp, synthetic construct
  source 1..20: mol_type = other DNA
acacctaaagcagcggttga

SEQ ID NO: 36 - DNA, 50 bp, synthetic construct
  source 1..50: mol_type = other DNA
ctctctctctctctctctctctctctctctacggacagcatcagtagtgt

SEQ ID NO: 37 - DNA, 50 bp, synthetic construct
  source 1..50: mol_type = other DNA
gtgtgtgtgtgtgtgtgtgtgtgtgtgtgtacggacagcatcagtagtgt

SEQ ID NO: 38 - DNA, 50 bp, synthetic construct
  source 1..50: mol_type = other DNA
atatatatatatatatatatatatatatatacggacagcatcagtagtgt

SEQ ID NO: 39 - DNA, 40 bp, synthetic construct
  source 1..40: mol_type = other DNA
ctctctctctctctctctctacggacagcatcagtagtgt

SEQ ID NO: 40 - DNA, 60 bp, synthetic construct
  source 1..60: mol_type = other DNA
ctctctctctctctctctctctctctctctctctctctctacggacagcatcagtagtgt

SEQ ID NO: 41 - DNA, 40 bp, synthetic construct
  source 1..40: mol_type = other DNA
gtgtgtgtgtgtgtgtgtgtacggacagcatcagtagtgt

SEQ ID NO: 42 - DNA, 60 bp, synthetic construct
  source 1..60: mol_type = other DNA
gtgtgtgtgtgtgtgtgtgtgtgtgtgtgtgtgtgtgtgtacggacagcatcagtagtgt

SEQ ID NO: 43 - DNA, 50 bp, synthetic construct
  source 1..50: mol_type = other DNA
acggacagcatcagtagtgtctctctctctctctctctctctctctctct

SEQ ID NO: 44 - DNA, 50 bp, synthetic construct
  source 1..50: mol_type = other DNA
acggacagcatcagtagtgtgtgtgtgtgtgtgtgtgtgtgtgtgtgtgt

SEQ ID NO: 45 - DNA, 50 bp, synthetic construct
  source 1..50: mol_type = other DNA
acggacagcatcagtagtgtatatatatatatatatatatatatatatat

SEQ ID NO: 46 - DNA, 50 bp, synthetic construct
  source 1..50: mol_type = other DNA
ctctctctctctctctctctctctctctcttcttggcaacctcattgagg

SEQ ID NO: 47 - DNA, 50 bp, synthetic construct
  source 1..50: mol_type = other DNA
gtgtgtgtgtgtgtgtgtgtgtgtgtgtgttcttggcaacctcattgagg

SEQ ID NO: 48 - DNA, 50 bp, synthetic construct
  source 1..50: mol_type = other DNA
tcttggcaacctcattgaggctctctctctctctctctctctctctctct

SEQ ID NO: 49 - DNA, 50 bp, synthetic construct
  source 1..50: mol_type = other DNA
tcttggcaacctcattgagggtgtgtgtgtgtgtgtgtgtgtgtgtgtgt

SEQ ID NO: 50 - DNA, 50 bp, synthetic construct; identical to SEQ ID NO: 49
  source 1..50: mol_type = other DNA

SEQ ID NO: 51 - DNA, 50 bp, synthetic construct
  source 1..50: mol_type = other DNA
gtgtgtgtgtgtgtgtgtgtgtgtgtgtgtatgttgagtgagagcggtga

SEQ ID NO: 52 - DNA, 50 bp, synthetic construct
  source 1..50: mol_type = other DNA
atgttgagtgagagcggtgactctctctctctctctctctctctctctct

SEQ ID NO: 53 - DNA, 50 bp, synthetic construct
  source 1..50: mol_type = other DNA
atgttgagtgagagcggtgagtgtgtgtgtgtgtgtgtgtgtgtgtgtgt

SEQ ID NO: 54 - DNA, 50 bp, synthetic construct
  source 1..50: mol_type = other DNA
ctctctctctctctctctctctctctctcttgaactgttgcgactacgtg

SEQ ID NO: 55 - DNA, 50 bp, synthetic construct
  source 1..50: mol_type = other DNA
gtgtgtgtgtgtgtgtgtgtgtgtgtgtgttgaactgttgcgactacgtg

SEQ ID NO: 56 - DNA, 50 bp, synthetic construct
  source 1..50: mol_type = other DNA
tgaactgttgcgactacgtgctctctctctctctctctctctctctctct

SEQ ID NO: 57 - DNA, 50 bp, synthetic construct
  source 1..50: mol_type = other DNA
tgaactgttgcgactacgtggtgtgtgtgtgtgtgtgtgtgtgtgtgtgt

SEQ ID NO: 58 - DNA, 50 bp, synthetic construct
  source 1..50: mol_type = other DNA
ctctctctctctctctctctctctctctctacgcgcaaacagtctgaaag

SEQ ID NO: 59 - DNA, 50 bp, synthetic construct
  source 1..50: mol_type = other DNA
gtgtgtgtgtgtgtgtgtgtgtgtgtgtgtacgcgcaaacagtctgaaag

SEQ ID NO: 60 - DNA, 50 bp, synthetic construct
  source 1..50: mol_type = other DNA
acgcgcaaacagtctgaaagctctctctctctctctctctctctctctct

SEQ ID NO: 61 - DNA, 50 bp, synthetic construct
  source 1..50: mol_type = other DNA
acgcgcaaacagtctgaaaggtgtgtgtgtgtgtgtgtgtgtgtgtgtgt

SEQ ID NO: 62 - DNA, 50 bp, synthetic construct
  source 1..50: mol_type = other DNA
ctctctctctctctctctctctctctctcttgcagcaaaacctgagtcac

SEQ ID NO: 63 - DNA, 50 bp, synthetic construct
  source 1..50: mol_type = other DNA
gtgtgtgtgtgtgtgtgtgtgtgtgtgtgttgcagcaaaacctgagtcac

SEQ ID NO: 64 - DNA, 50 bp, synthetic construct
  source 1..50: mol_type = other DNA
tgcagcaaaacctgagtcacctctctctctctctctctctctctctctct

SEQ ID NO: 65 - DNA, 50 bp, synthetic construct
  source 1..50: mol_type = other DNA
tgcagcaaaacctgagtcacgtgtgtgtgtgtgtgtgtgtgtgtgtgtgt

SEQ ID NO: 66 - DNA, 51 bp, synthetic construct
  source 1..51: mol_type = other DNA
ctctctctctctctctctctctctctctctagagtaaacgtaaaaagaagg

SEQ ID NO: 67 - DNA, 51 bp, synthetic construct
  source 1..51: mol_type = other DNA
gtgtgtgtgtgtgtgtgtgtgtgtgtgtgtagagtaaacgtaaaaagaagg

SEQ ID NO: 68 - DNA, 51 bp, synthetic construct
  source 1..51: mol_type = other DNA
agagtaaacgtaaaaagaaggctctctctctctctctctctctctctctct

SEQ ID NO: 69 - DNA, 51 bp, synthetic construct
  source 1..51: mol_type = other DNA
agagtaaacgtaaaaagaagggtgtgtgtgtgtgtgtgtgtgtgtgtgtgt

SEQ ID NO: 70 - DNA, 50 bp, synthetic construct
  source 1..50: mol_type = other DNA
ctctctctctctctctctctctctctctctgatcaggaactctagaagaa

SEQ ID NO: 71 - DNA, 50 bp, synthetic construct
  source 1..50: mol_type = other DNA
gtgtgtgtgtgtgtgtgtgtgtgtgtgtgtgatcaggaactctagaagaa

SEQ ID NO: 72 - DNA, 50 bp, synthetic construct
  source 1..50: mol_type = other DNA
gatcaggaactctagaagaactctctctctctctctctctctctctctct

SEQ ID NO: 73 - DNA, 50 bp, synthetic construct
  source 1..50: mol_type = other DNA
gatcaggaactctagaagaagtgtgtgtgtgtgtgtgtgtgtgtgtgtgt

SEQ ID NO: 74 - DNA, 50 bp, synthetic construct
  source 1..50: mol_type = other DNA
ctctctctctctctctctctctctctctctgtcctggttgaatgcgaaca

SEQ ID NO: 75 - DNA, 50 bp, synthetic construct
  source 1..50: mol_type = other DNA
gtgtgtgtgtgtgtgtgtgtgtgtgtgtgtgtcctggttgaatgcgaaca

SEQ ID NO: 76 - DNA, 50 bp, synthetic construct
  source 1..50: mol_type = other DNA
gtcctggttgaatgcgaacactctctctctctctctctctctctctctct

SEQ ID NO: 77 - DNA, 50 bp, synthetic construct
  source 1..50: mol_type = other DNA
gtcctggttgaatgcgaacagtgtgtgtgtgtgtgtgtgtgtgtgtgtgt

SEQ ID NO: 78 - DNA, 50 bp, synthetic construct
  source 1..50: mol_type = other DNA
ctctctctctctctctctctctctctctcttggaacccaataggcacact

SEQ ID NO: 79 - DNA, 50 bp, synthetic construct
  source 1..50: mol_type = other DNA
gtgtgtgtgtgtgtgtgtgtgtgtgtgtgttggaacccaataggcacact

SEQ ID NO: 80 - DNA, 50 bp, synthetic construct
  source 1..50: mol_type = other DNA
tggaacccaataggcacactctctctctctctctctctctctctctctct

SEQ ID NO: 81 - DNA, 50 bp, synthetic construct
  source 1..50: mol_type = other DNA
tggaacccaataggcacactgtgtgtgtgtgtgtgtgtgtgtgtgtgtgt

SEQ ID NO: 82 - DNA, 50 bp, synthetic construct
  source 1..50: mol_type = other DNA
ctctctctctctctctctctctctctctctacacctaaagcagcggttga

SEQ ID NO: 83 - DNA, 50 bp, synthetic construct
  source 1..50: mol_type = other DNA
gtgtgtgtgtgtgtgtgtgtgtgtgtgtgtacacctaaagcagcggttga

SEQ ID NO: 84 - DNA, 50 bp, synthetic construct
  source 1..50: mol_type = other DNA
acacctaaagcagcggttgactctctctctctctctctctctctctctct

SEQ ID NO: 85 - DNA, 50 bp, synthetic construct
  source 1..50: mol_type = other DNA
acacctaaagcagcggttgagtgtgtgtgtgtgtgtgtgtgtgtgtgtgt